(19) United States
(12) Patent Application Publication
(10) Pub. No.: US 2021/0009549 A1
(43) Pub. Date: Jan. 14, 2021

(54) CANNABINOID DERIVATIVES AND CONJUGATES AND USES THEREOF

(71) Applicant: BEETLEBUNG PHARMA LTD., Katzrin (IL)

(72) Inventors: Prakash JAGTAP, North Andover, MA (US); Dana SHOKEN, Kfar Kama (IL); Shlomit AVIDAN- SHLOMOVICH, Haifa (IL); Andrew Lurie SALZMAN, West Tisbury, MA (US)

(21) Appl. No.: 16/969,942

(22) PCT Filed: Feb. 13, 2019
(86) PCT No.: PCT/IL2019/050172

§ 371 Date: Aug. 13, 2020

Related U.S. Application Data
(60) Provisional application No. 62/629,796, filed on Feb. 13, 2018.

(51) Int. Cl. C07D 311/78; C07C 39/23

(52) U.S. Cl. CPC C07D 311/78; C07C 39/23

(57) ABSTRACT

Cannavinoid derivatives, such as cannabidiol (CBD), desoxy-CBD, and desoxy-Δ⁹-tetrahydrocannabinol (desoxy-THC) derivatives, or an enantiomer, diastereomer, racemate, or pharmaceutically acceptable salt thereof, are useful for neuroprotection, treating pain, or treating a disease associated with alpha-1 glycine receptor (α1GlyR) and/or alpha-3 glycine receptor (α3GlyR) deficiency. Drug conjugates of the derivatives can be made.

### TECHNICAL FIELD

The present invention relates to cannabinoid derivatives, more specifically cannabidiol (CBD), desoxy-CBD, and Δ⁹-tetrahydrocannabinol (THC) derivatives, and drug conjugates thereof, and to uses thereof.

Abbreviations: ACN, acetonitrile; DAST, diethylamino sulfur trifluoride; DCC, N,N′-dicyclohexylcarbodiimide; DCM, dichloromethane; DIBAL, diisobutylaluminum; DMAP, 4-dimethylaminopyridine; EGTA, ethylene glycol-bis(β-aminoethyl ether)-N,N,N′,N′-tetraacetic acid; HEPES, 4-(2-hydroxyethyl)-1-piperazineethanesulfonic acid; p-TSA, p-toluenesulfonic acid; TLC, thin-layer chromatography.

### BACKGROUND ART

Cannabinoids and cannabinoid prodrugs can be used to treat medical conditions responsive to cannabinoids, including pain and neuroprotection. These medical conditions are both acute and chronic, necessitating cannabinoid molecules that may be delivered parenterally for acute conditions, and therefore will optimally be water soluble; or orally for chronic conditions, and therefore will optimally have increased bioavailability. The diversity of biological mechanisms that are responsible for these medical conditions may also benefit from cannabinoid agents that modulate a broader range of biological targets than can be attributed to the actions of a cannabinoid alone.

Xiong et al. (2011) disclose that the serine residue at position 296 in the glycine receptor (GlyR), an important target for nociceptive regulation at the spinal level, is critical for the potentiation of IGl_{y} by THC. As shown, the polarity of the serine residue and the hydroxyl groups of THC are critical for THC potentiation. The cannabinoid-induced analgesia is absent in mice lacking alpha-3 glycine receptors (α3GlyRs) but not in those lacking CB1 and CB2 receptors.

Xiong et al. (2012) discloses that systemic and intrathecal administration of CBD, or certain derivatives thereof including dehydroxylcannabidiol (DH-CBD, also referred to as desoxy CBD), significantly suppresses chronic inflammatory and neuropathic pain without causing apparent analgesic tolerance in rodents, and that while the analgesic potency of those cannabinoids is positively correlated with cannabinoid potentiation of the α3 GlyRs, it is neither correlated with their binding affinity for CB1 and CB2 receptors nor with their psychoactive side effects.

Xiong et al. (2014) discloses that DH-CBD, a nonpsychoactive cannabinoid, selectively rescues impaired presynaptic GlyR activity and diminished glycine release in the brainstem and spinal cord of hyperekplexic mutant mice, suggesting that presynaptic a GlyRs emerge as a potential therapeutic target for dominant hyperekplexia disease and other diseases with GlyR deficiency.

Pop et al. (1999) disclose trialkylammonium acetoxymethyl esters of dexanabinol. As stated, most of the prodrugs synthesized were soluble and relatively stable in water, while rapidly hydrolyzed in human plasma; and distribution studies in rats indicated that peak concentrations of drug both in blood and brain were rapidly achieved after IV administration of a selected prodrug.

Kinney et al. (2016) disclose a series of side chain modified resorcinols designed for greater hydrophilicity and “drug likeness”, while varying certain parameters within the pendent group. As stated, some of those agents prevented damage to hippocampal neurons induced by ammonium acetate and ethanol at clinically relevant concentrations, and one of them (identified therein as “KLS-13019”) was 50-fold more potent and >400-fold safer than CBD, and exhibited an in vitro profile consistent with improved oral bioavailability.

### SUMMARY OF INVENTION

In one aspect, the present invention provides a cannabinoid compound of the formula I:

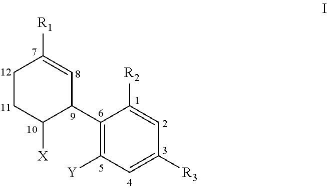

wherein:

X is the radical

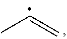

and Y is H, —OH, —OH, —OR₄, or R₄; or

X is the radical

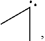

and Y is —O—, and together with X and the carbon atoms to which they are attached form a dihydropyran ring,

or an enantiomer, diastereomer, racemate, or pharmaceutically acceptable salt thereof,

wherein:

R₁ is (C₁-C₃)alkyl, (C₁-C₃)haloalkyl, —(C₁-C₃)alkylene-OH, —(C₁-C₃)alkylene-COOH, —(C₁-C₃)alkylene-O—(C₁-C₁₂)alkyl, —(C₁-C₃)alkylene-O—C(O)—(C₁-C₁₂)alkyl, —(C₁-C₃)alkylene-C(O)—O—(C₁-C₁₂)alkyl, —COOH, R₆, or —(C₁-C₃)alkylene-R₆;

R₂ is H, —OH, —OR₄, or R₄;

R₃ is —OH, —OR₅, or R₅;

R₄ and R₅ each independently is (C₁-C₁₂)alkyl, (C₁-C₁₂)haloalkyl, (C₂-C₁₂)alkenyl, (C₂-C₁₂)alkynyl, (C₃-C₈)cycloalkyl, (C₃-C₈)cycloalkenyl, (C₃-C₈)cycloalkylene-(C₁-C₁₂)alkyl, (C₁-C₁₂)alkylene-(C₃-C₈)cyclo alkyl, —C(O)—(C₁-C₁₂)alkyl, —C(O)—(C₁-C₁₂)haloalkyl, —C(O)—(C₂-C₁₂)alkenyl, —C(O)—(C₂-C₁₂)alkynyl, —C(O)—(C₃-C₈)cycloalkyl, —C(O)—(C₃-C₈)cycloalkenyl, non-aromatic (C₃-C₈)heterocyclyl, bridged (C₆-C₁₄)bicycloalkyl, bridged (C₈-C₁₆)tricycloalkyl, R₆, or the radical of the formula II:

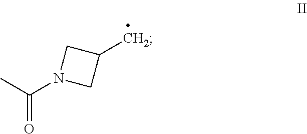

and

R₆ each independently is a drug selected from naproxen, ibuprofen, aspirin, betaine (trimethyl glycine), an opiate, an inducible nitric oxide synthase (iNOs) inhibitor, a PARP inhibitor, or a derivative thereof, linked directly or via a linker,

provided that: (i) Y is H, but excluding the compound wherein R₂ is H; or wherein R₁ is CH₃, R₂ is —OH, and R₃ is n-pentyl (DH-CBD); or (ii) Y is —O—; and R₂ is H or R₄, but excluding the compound wherein R₁ is CH₃, R₂ is H, and R₃ is n-pentyl (desoxy-THC); or (iii) Y is neither H nor —O—; R₂ is not H; and (a) R₁ is —(C₁-C₃)alkylene-R₆; or (b) R₂ is R₄ wherein R₄ is R₆; or (c) R₃ is R₅ wherein R₅ is R₆; or (d) Y is R₄ wherein R₄ is R₆.

Specific novel compounds of the formula I described in the specification are herein identified by the Arabic numbers 101-153 in bold, and their full chemical structures are shown in Tables 3-5 hereinafter.

In another aspect, the present invention provides a cannabinoid compound of the formula III:

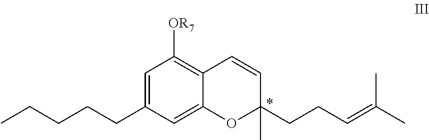

or an enantiomer, diastereomer, racemate, or pharmaceutically acceptable salt thereof,

wherein R₇ is a drug selected from naproxen, ibuprofen, aspirin, betaine, an opiate, an iNOs inhibitor, a PARP inhibitor, or a derivative thereof, linked directly or via a linker.

In a further aspect, the present invention provides a pharmaceutical composition comprising a cannabinoid compound of the formula I or III as defined above, or an enantiomer, diastereomer, racemate, or pharmaceutically acceptable salt thereof, and a pharmaceutically acceptable carrier. In one particular such aspect, the pharmaceutical composition disclosed herein comprises a compound of the formula I as defined above. In another particular such aspect, the pharmaceutical composition disclosed herein comprises a compound of the formula III as defined above. The compounds and pharmaceutical compositions of the present invention are useful for providing neuroprotection, treating pain, or treating a disease associated with GlyR deficiency such as hyperekplexia disease.

In yet a further aspect, the present invention relates to a cannabinoid compound of the formula I or III as defined above, or an enantiomer, diastereomer, racemate, or pharmaceutically acceptable salt thereof, for use in providing neuroprotection, treating pain, or treating a disease associated with GlyR deficiency such as hyperekplexia disease.

In still a further aspect, the present invention relates to use of a cannabinoid compound of the formula I or III as defined above, or an enantiomer, diastereomer, racemate, or pharmaceutically acceptable salt thereof, for the preparation of a pharmaceutical composition for providing neuroprotection, treating pain, or treating a disease associated with GlyR deficiency such as hyperekplexia disease.

In another aspect, the present invention relates to a method for providing neuroprotection, treating pain, or treating a disease associated with GlyR deficiency such as hyperekplexia disease, in an individual in need thereof, comprising administering to said individual an effective amount of a cannabinoid compound of the formula I or III as defined above, or an enantiomer, diastereomer, racemate, or pharmaceutically acceptable salt thereof.

### DETAILED DESCRIPTION

The present invention relates to a series of cannabinoid molecules that may be useful for neuroprotection, treating pain, or treating a disease associated with alpha-1 glycine receptor (α1GlyR) and/or alpha-3 glycine receptor (α3GlyR) deficiency. Activation of these receptors inhibits nociceptive transmission and thus exerts an analgesic effect. Some of the compounds disclosed herein are in fact conjugates, wherein the cannabinoid molecule is conjugated to a second analgesic molecule, such as a nonsteroidal anti-inflammatory drug (NSAID) or an opiate, in order to provide two complementary independent and non-overlapping analgesic effects. In those conjugates, the cannabinoid molecule and the second analgesic molecule are connected via an ester linkage that is susceptible to hydrolysis by enzymes within the body, and it is therefore expected that administration of the conjugates will result in the separation of the two molecules in vivo.

In one aspect, the present invention thus provides a cannabinoid compound of the formula I as defined above, or an enantiomer, diastereomer, racemate, or pharmaceutically acceptable salt thereof.

In another aspect, the present invention provides a cannabinoid compound of the formula III as defined above, or an enantiomer, diastereomer, racemate, or pharmaceutically acceptable salt thereof.

The term “alkyl” as used herein typically means a linear or branched saturated hydrocarbon radical having 1-12 carbon atoms and includes, e.g., methyl, ethyl, n-propyl, isopropyl, n-butyl, sec-butyl, isobutyl, tert-butyl, n-pentyl, isopentyl, neopentyl, 2,2-dimethylpropyl, n-hexyl, isohexyl, n-heptyl, 1,1-dimethylpentyl, 1,1-dimethylbutyl, 2,2-dimethylbutyl, 2-ethylbutyl, 1,1-dimethylheptyl (1,1-DMH), 1,2-DMH, n-octyl, n-nonyl, n-decyl, n-undecyl, n-dodecyl, and the like. Preferred are (C₁-C₈)alkyl groups, more preferably (C₁-C₃)alkyl groups, most preferably methyl and ethyl. The terms “alkenyl” and “alkynyl” typically mean linear or branched hydrocarbon radicals having 2-12 carbon atoms and one double or triple bond, respectively, and include ethenyl, propenyl, 3-buten-1-yl, 2-ethenylbutyl, 3-octen-1-yl, 3-nonenyl, 3-decenyl, and the like, and propynyl, 2-butyn-1-yl, 3-pentyn-1-yl, 3-hexynyl, 3-octynyl, 4-decynyl, and the like. C₂-C₆ alkenyl and alkynyl radicals are preferred, more preferably C₂-C₄ alkenyl and alkynyl.

The term “haloalkyl” as used herein typically means an alkyl as defined hereinabove, which is substituted with one or more, e.g., one, two or three, halogens each independently being selected from fluoro, chloro, bromo, or iodo. Preferred haloalkyls are alkyls substituted with one halogen such as fluoro or chloro.

The term “alkylene” typically means a divalent linear or branched hydrocarbon radical having 1-6 carbon atoms and includes, e.g., methylene, ethylene, propylene, butylene, 2-methylpropylene, pentylene, 2-methylbutylene, hexylene, and the like. Preferred are (C₁-C₃)alkylene, more preferably methylene or ethylene.

The term “cycloalkyl” as used herein means a cyclic hydrocarbyl group having 3-8 carbon atoms such as cyclopropyl, cyclobutyl, cyclopentyl, cyclohexyl, cycloheptyl, cyclooctyl, and the like. Preferred are (C₅-C₇)cycloalkyls. The term “cycloalkenyl” as used herein means a cyclic or bicyclic hydrocarbyl group having 3-8 carbon atoms such as cyclopropenyl (e.g., 2-cyclopropen-1-yl), cyclobutenyl (e.g., 2-cyclobuten-1-yl), cyclopentenyl (e.g., 2-cyclopenten-1-yl, and 3-cyclopenten-1-yl), cyclohexenyl (e.g., 2-cyclohexen-1-yl, and 3-cyclohexen-1-yl), and the like.

The term “bridged (C₆-C₁₄)bicycloalkyl” as used herein refers to a saturated (non-aromatic) cyclic hydrocarbon radicals formed by two fused rings of six to fourteen carbon atoms. Examples of such radicals include bicyclo[2.2.1]heptyl, bicyclo[3.2.0]heptyl, bicyclo[4.4.0]decyl, bicyclo[3.3.0]octyl, bicyclo[3.2.1]octyl, bicyclo[1.1.1]pentyl, bicyclo[4.3.0]nonyl, and 8-methylbicyclo[4.3.0]nonyl.

The term “bridged (C₈-C₁₆)tricycloalkyl” as used herein refers to a saturated (non-aromatic) cyclic hydrocarbon radicals formed by three fused rings of eight to sixteen carbon atoms. Example of such radicals include tricyclo[2.2.1.0(2,6)]heptyl, tricyclo[5.2.1.0(2,6)]decyl, tricyclo(4,3,0,0)nonyl, tricyclo[3.1.1.0(6,7)]heptyl, trimethylenenorbornyl, tricyclo[6.2.1.13,6]dodecyl, tricyclo[6.4.0.0(2,7)]dodecyl, exo-tricyclo[5.2.1.0(2.6)]decyl, and adamantly.

The term “non-aromatic heterocyclic ring” denotes a mono- or poly-cyclic non-aromatic ring of 3-8 atoms containing at least one carbon atom and one to three heteroatoms selected from oxygen, sulfur (optionally oxidized), or nitrogen, which may be saturated or unsaturated, i.e., containing at least one unsaturated bond. Preferred are 5- or 6-membered heterocyclic rings. Non-limiting examples of non-aromatic heterocyclic ring include azetidine, pyrrolidine, piperidine, morpholine, thiomorpholine, piperazine, oxazolidine, thiazolidine, imidazolidine, oxazoline, thiazoline, imidazoline, dioxole, dioxolane, dihydrooxadiazole, pyran, dihydropyran, tetrahydropyran, thiopyran, dihydrothiopyran, tetrahydrothiopyran, 1-oxidotetrahydro thiopyran, 1,1-dioxidotetrahydrothiopyran, tetrahydrofuran, pyrazolidine, pyrazoline, tetrahydropyrimidine, dihydrotriazole, tetrahydrotriazole, azepane, dihydropyridine, tetrahydropyridine, and the like. The term “non-aromatic heterocyclyl” as used herein refers to any univalent radical derived from a non-aromatic heterocyclic ring as defined herein by removal of hydrogen from any ring atom. Examples of such radicals include, without limiting, piperidino, 4-morpholinyl, and pyrrolidinyl.

In certain embodiments, the present invention provides a compound of the formula I, wherein R₁ is (C₁-C₃)alkyl, (C₁-C₃)haloalkyl, —(C₁-C₃)alkylene-OH, —(C₁-C₃)alkylene-O—C(O)—(C₁-C₁₂)alkyl, or —(C₁-C₃)alkylene-R₆. Particular such compounds are those wherein R₁ is (C₁-C₂)alkyl, —(C₁-C₂)alkylene-OH, —(C₁-C₂)alkylene-O—C(O)—(C₁-C₁₂)alkyl, or —(C₁-C₂)alkylene-R₆. In more particular such compounds, R₁ is —CH₃, —CH₂F, —CH₂—OH, —CH₂—O—C(O)—(C₁-C₁₂)alkyl, e.g., —CH₂—O—C(O)—(C₁-C₈)alkyl or —CH₂—O—C(O)—(C₁-C₄)alkyl, or —CH₂—R₆.

In certain embodiments, the present invention provides a compound of the formula I, wherein R₂ is H, —OH, —OR₄, or R₄; and R₄ is (C₁-C₁₂)alkyl, e.g., (C₁-C₈)alkyl or (C₁-C₄)alkyl, —C(O)—(C₁-C₁₂)alkyl, e.g., —C(O)—(C₁-C₈)alkyl or —C(O)—(C₁-C₄)alkyl, (C₃-C₈)cycloalkylene-(C₁-C₁₂)alkyl, R₆ linked directly or via a linker, or the radical of the formula II. Particular such compounds are those wherein (i) R₂ is H, or —OH; (ii) R₂ is —OR₄; and R₄ is —C(O)—(C₁-C₁₂)alkyl, e.g., —C(O)—(C₁-C₈)alkyl or —C(O)—(C₁-C₄)alkyl; or (iii) R₂ is R₄; and R₄ is R₆ linked directly or via a linker.

In certain embodiments, the present invention provides a compound of the formula I, wherein R₃ is —OH, —OR₅, or R₅; and R₅ is (C₁-C₁₂)alkyl, e.g., (C₁-C₈)alkyl or (C₁-C₄)alkyl, —C(O)—(C₁-C₁₂)alkyl, e.g., —C(O)—(C₁-C₈)alkyl or —C(O)—(C₁-C₄)alkyl, (C₃-C₈)cycloalkylene-(C₁-C₁₂)alkyl, R₆ linked directly or via a linker, or the radical of the formula II. Particular such compounds are those wherein R₃ is R₅; and R₅ is (C₁-C₁₂)alkyl, e.g., (C₁-C₈)alkyl or (C₁-C₄)alkyl, (C₃-C₈)cycloalkylene-(C₁-C₁₂)alkyl, R₆ linked directly or via a linker, or the radical of the formula II.

In certain embodiments, the present invention provides a compound of the formula I, wherein R₁ is (C₁-C₃)alkyl, e.g., (C₁-C₂)alkyl, (C₁-C₃)haloalkyl, e.g., (C₁-C₂)haloalkyl, —(C₁-C₃)alkylene-OH, e.g., —(C₁-C₂)alkylene-OH, —(C₁-C₃)alkylene-O—C(O)—(C₁-C₁₂)alkyl, e.g., —(C₁-C₂)alkylene-O—C(O)—(C₁-C₁₂)alkyl, or —(C₁-C₃)alkylene-R₆, e.g., —(C₁-C₂)alkylene-R₆; R₂ is H, —OH, —OR₄, or R₄; R₃ is —OH, —OR₅, or R₅; and R₄ and R₅ each independently is (C₁-C₁₂)alkyl, e.g., (C₁-C₈)alkyl or (C₁-C₄)alkyl, —C(O)—(C₁-C₁₂)alkyl, e.g., —C(O)—(C₁-C₈)alkyl or —C(O)—(C₁-C₄)alkyl, (C₃-C₈)cycloalkylene-(C₁-C₁₂)alkyl, R₆ linked directly or via a linker, or the radical of the formula II. In some particular such embodiments, 121 is —CH₃, —CH₂F, —CH₂—OH, —CH₂—O—C(O)—(C₁-C₁₂)alkyl, e.g., —CH₂—O—C(O)—(C₁-C₈)alkyl or —CH₂—O—C(O)—(C₁-C₄)alkyl, or —CH₂—R₆; R₂ is H, or —OH; R₃ is R₅; R₅ is (C₁-C₁₂)alkyl, e.g., (C₁-C₈)alkyl or (C₁-C₄)alkyl, (C₃-C₈)cycloalkylene-(C₁-C₁₂)alkyl, R₆, or the radical of the formula II; and R₆ each independently is a drug linked directly or via a linker. In other particular such embodiments, 121 is —CH₃, —CH₂F, —CH₂—OH, —CH₂—O—C(O)—(C₁-C₁₂)alkyl, e.g., —CH₂—O—C(O)—(C₁-C₈)alkyl or —CH₂—O—C(O)—(C₁-C₄)alkyl, or —CH₂—R₆; R₂ is —OR₄; R₃ is R₅; R₄ is —C(O)—(C₁-C₁₂)alkyl, e.g., —C(O)—(C₁-C₈)alkyl or —C(O)—(C₁-C₄)alkyl; R₅ is (C₁-C₁₂)alkyl, e.g., (C₁-C₈)alkyl or (C₁-C₄)alkyl, (C₃-C₈)cycloalkylene-(C₁-C₁₂)alkyl, R₆, or the radical of the formula II; and R₆ each independently is said drug linked directly or via a linker. In further particular such embodiments, 121 is —CH₃, —CH₂F, —CH₂—OH, —CH₂—O—C(O)—(C₁-C₁₂)alkyl, e.g., —CH₂—O—C(O)—(C₁-C₈)alkyl or —CH₂—O—C(O)—(C₁-C₄)alkyl, or —CH₂—R₆; R₂ is R₄; R₃ is R₅; R₄ is R₆; R₅ is (C₁-C₁₂)alkyl, e.g., (C₁-C₈)alkyl or (C₁-C₄)alkyl, (C₃-C₈)cycloalkylene-(C₁-C₁₂)alkyl, R₆, or the radical of the formula II; and R₆ each independently is a drug linked directly or via a linker.

The compounds of the formulae I and III are cannabinoid prodrugs, wherein a drug is optionally linked to the cannabinoid structure via a functional group of said drug, either directly or via a linker. The drug moiety conjugated to the cannabinoid structure is represented by the group R₆ in the general formula I, and may be linked, directly or indirectly, to any one of the carbon atoms at positions 1, 3, 5 or 7 in the formula I; or by the group R₇ in the general formula III. In certain embodiments, the compound of the formula I according to any one of the embodiments above is a cannabinoid structure conjugated to a drug moiety, i.e., a compound of the formula I wherein one of the carbon atoms at positions 1, 3, 5 and 7 is linked, either directly or indirectly, to a drug moiety. In other embodiments, the compound of the formula I according to any one of the embodiments above is a cannabinoid structure conjugated to more than one drug moieties, e.g., a compound of the formula I wherein two or three of the carbon atoms at positions 1, 3, 5 and 7 each is linked, directly or indirectly, to a drug moiety. Particular such cannabinoid prodrugs include, e.g., compounds of the formula I wherein each one of the carbon atoms at positions 1 and 3; 1 and 5; 1 and 7; 3 and 5; 3 and 7; 5 and 7; 1, 3 and 5; 1, 3 and 7; or 3, 5 and 7, is linked, either directly or indirectly, to a drug moiety.

Examples of drugs that may be conjugated to the cannabinoid structure in the formula I or III include, without being limited to, naproxen (naprosyn); ibuprofen; aspirin; betaine (trimethyl glycine); an opiate such as codeine, dihydrocodeine, diamorphine, buprenorphine, methadone, fentanyl, hydromorphone, oxycodone, pethidine, morphine, dextropropoxyphene, and tramadol; a poly(ADP-ribose) polymerase (PARP) inhibitor such as olaparib, veliparib, veliparib acetate, rucaparib, talazoparib, PJ-34 (CAS Number: 344458-15-7), niraparib, and INO-1001; an iNOs inhibitor such as N-[[3-(Aminomethyl)phenyl]methyl]-ethanimidamide dihydrochloride (1400W; CAS Number: 214358-33-5), N⁶-(1-Iminoethyl)-L-lysine hydrochloride (L-NIL; CAS Number: 150403-89-7), N⁵-(1-Iminoethyl)-L-ornithine dihydrochloride (L-NIO; CAS Number: 159190-44-0), and (2S)-2-amino-4-[(2-ethanimidamidoethyl)sulfanyl]butanoic acid (GW274150; CAS Number: 210354-22-6); or a derivative thereof (see structures in Table 1).

TABLE 1
Specific drugs referred to herein

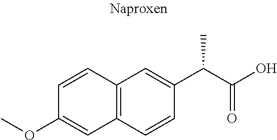 [SMILES: COc1ccc2cc([C@H](C)C(=O)O)ccc2c1]

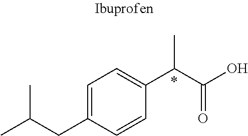

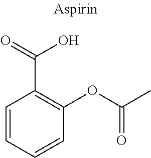

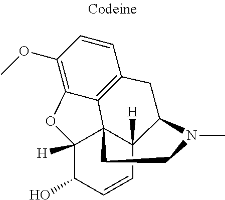

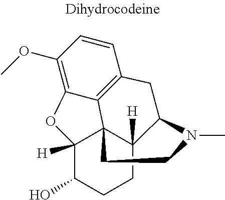

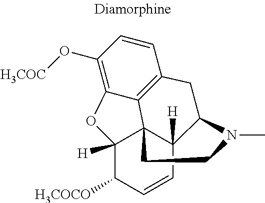

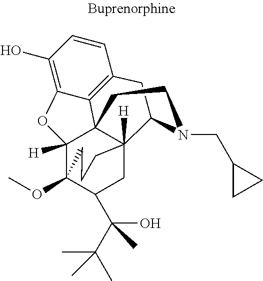

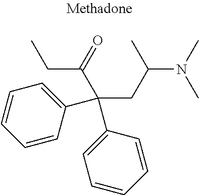 [SMILES: CCC(=O)C(CC(C)N(C)C)(c1ccccc1)c1ccccc1]

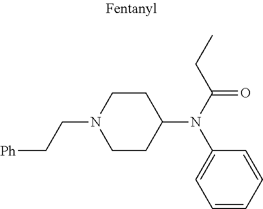 [SMILES: CCC(=O)N(c1ccccc1)C1CCN(CCc2ccccc2)CC1]

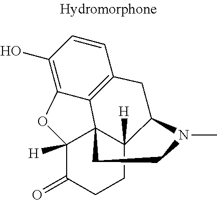

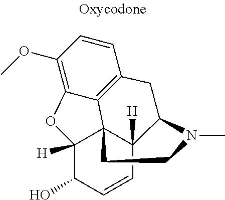

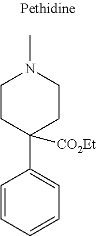 [SMILES: CCOC(=O)C1(c2ccccc2)CCN(C)CC1]

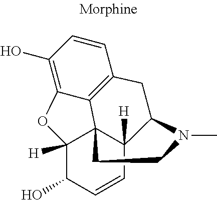

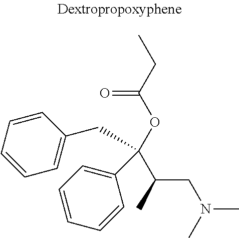 [SMILES: CCC(=O)O[C@](Cc1ccccc1)(c1ccccc1)[C@H](C)CN(C)C]

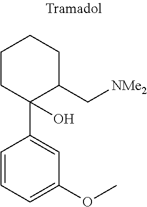 [SMILES: COc1cccc(C2(O)CCCCC2CN(C)C)c1]

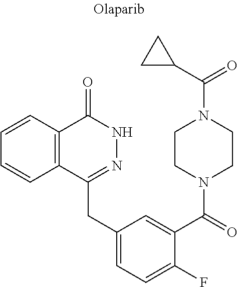 [SMILES: O=C(c1cc(Cc2n[nH]c(=O)c3ccccc23)ccc1F)N1CCN(C(=O)C2CC2)CC1]

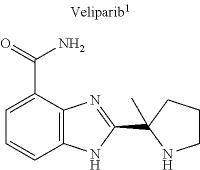

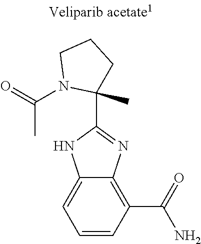 [SMILES: CC(=O)N1CCC[C@]1(C)c1nc2c(C(N)=O)cccc2[nH]1]

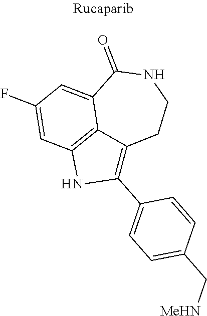 [SMILES: CNCc1ccc(-c2[nH]c3cc(F)cc4c3c2CCNC4=O)cc1]

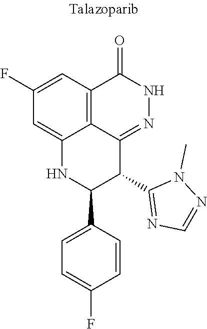 [SMILES: Cn1ncnc1[C@H]1c2n[nH]c(=O)c3cc(F)cc(c23)N[C@@H]1c1ccc(F)cc1]

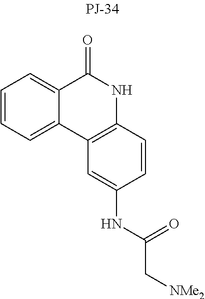 [SMILES: CN(C)CC(=O)Nc1ccc2[nH]c(=O)c3ccccc3c2c1]

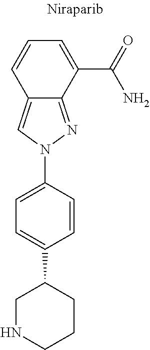 [SMILES: NC(=O)c1cccc2cn(-c3ccc([C@@H]4CCCNC4)cc3)nc12]

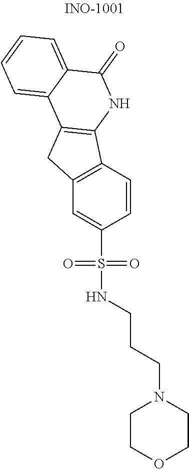 [SMILES: O=c1[nH]c2c(c3ccccc13)Cc1cc(S(=O)(=O)NCCCN3CCOCC3)ccc1-2]

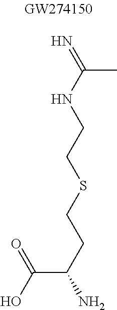

¹Veliparib derivatives include, e.g., veliparib-like compounds wherein one or
more of the hydrogen atoms of the amino- and/or secondary amino groups, is
replaced by a linear or branched alkyl each independently selected from, e.g.,
methyl, ethyl, n-propyl, or isopropyl.
²Betaine (trimethyl glycine) derivatives include, e.g., betaine-like compounds
wherein one or more of the methyl groups is replaced by a longer linear or
branched alkyl each independently selected from, e.g., ethyl, n-propyl, isopropyl,
or butyl; or two of said alkyl groups, together with the nitrogen atom to which
they are attached, form a 5-7 membered cyclic amine.

In certain embodiments, the drug (R₆ in the formula I, or R₇ in the formula III) is linked directly, e.g., through a functional group such as a carboxyl, amino, or methyl group, thereof. A non-limiting example of a drug that can be linked directly is veliparib, or a derivative thereof, which might be linked through the methyl group thereof.

In other embodiments, the drug (R₆ in the formula I, or R₇ in the formula III) is linked via a linker, e.g., through a functional group such as a carboxyl, amino, or methyl group, thereof. According to the present invention, suitable linkers are those having a first functional group capable of linking to the formula I or III, and a second functional group capable of linking to a functional group, e.g., a carboxyl, amino, or methyl group, of the drug. Certain particular such linkers have an ester group for linking to the formula I or III, and a methylene group for linking to a functional group of the drug, e.g., a linker of the formula —O—C(O)—(CH₂)ₙ—C(O)—O—CH₂—, wherein n is an integer of 1-8, preferably 1, 2, or 3, as exemplified herein. Such linkers can be used for linking, e.g., codeine, dihydrocodeine, diamorphine, hydromorphone, oxycodone, pethidine, morphine, and dextropropoxyphene through the nitrogen atom thereof; niraparib through the nitrogen atom of the piperidine ring; and PJ34, methadone, and tramadol through the dimethylamino group thereof. Other particular such linkers have an ester group for linking to the formula I or III, and an additional ester group for linking to a hydroxyl group of the drug, e.g., a linker of the formula —O—C(O)—(CH₂)ₙ—C(O)—O—, wherein n is an integer of 1-8, preferably 1, 2, or 3. Such linkers can be used for linking, e.g., codeine, dihydrocodeine, buprenorphine, hydromorphone, oxycodone, morphine and tramadol through the hydroxyl group thereof.

In certain embodiments, the present invention provides a compound of the formula I according to any one of the embodiments above, wherein Y is H, i.e., a desoxy-CBD derivative of the formula Ia in Table 2. Particular such compounds are those wherein (i) R₂ is —OH (formula Ia-1 in Table 2); (ii) R₂ is —OR₄; and R₄ is —C(O)—(C₁-C₁₂)alkyl, e.g., —C(O)—(C₁-C₈)alkyl or —C(O)—(C₁-C₄)alkyl (formula Ia-2 in Table 2); or (iii) R₂ is R₄; and R₄ is (C₁-C₁₂)alkyl, e.g., (C₁-C₈)alkyl or (C₁-C₄)alkyl, R₆, or the radical of the formula II (formula Ia-3 in Table 2). More particular such compounds are those wherein R₁ is —CH₃, —CH₂F, —CH₂—OH, —CH₂—O—C(O)—(C₁-C₁₂)alkyl, e.g., —CH₂—O—C(O)—(C₁-C₈)alkyl or —CH₂—O—C(O)—(C₁-C₄)alkyl, or —CH₂—R₆; R₃ is R₅; and R₅ is (C₁-C₁₂)alkyl, e.g., (C₁-C₈)alkyl, (C₃-C₈)cycloalkylene-(C₁-C₁₂)alkyl, R₆, or the radical of the formula II.

In other embodiments, the present invention provides a compound of the formula I according to any one of the embodiments above, wherein Y is —OH; —OR₄; or R₄ wherein R₄ is R₆, i.e., a CBD derivative of the formula Ib in Table 2. Particular such compounds are those wherein (i) R₂ is —OH (formula Ib-1 in Table 1); (ii) R₂ is —OR₄; and R₄ is —C(O)—(C₁-C₁₂)alkyl, e.g., —C(O)—(C₁-C₈)alkyl or —C(O)—(C₁-C₄)alkyl (formula Ib-2 in Table 1); or (iii) R₂ is R₄; and R₄ is (C₁-C₁₂)alkyl, e.g., (C₁-C₈)alkyl or (C₁-C₄)alkyl, R₆, or the radical of the formula II (formula Ib-3 in Table 2). More particular such compounds are those wherein R₁ is —CH₃, —CH₂F, —CH₂—OH, —CH₂—O—C(O)—(C₁-C₁₂)alkyl, e.g., —CH₂—O—C(O)—(C₁-C₈)alkyl or —CH₂—O—C(O)—(C₁-C₄)alkyl, or —CH₂—R₆; R₃ is R₅; and R₅ is (C₁-C₁₂)alkyl, e.g., (C₁-C₈)alkyl, (C₃-C₈)cycloalkylene-(C₁-C₁₂)alkyl, R₆, or the radical of the formula II.

TABLE 2
Particular structures of the formula I referred to herein

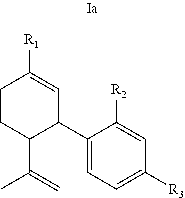

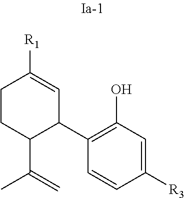

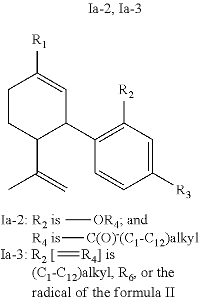

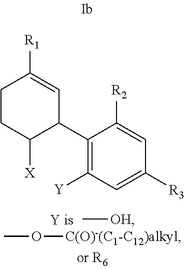

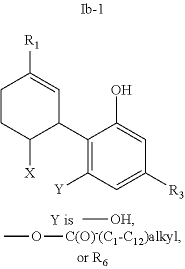

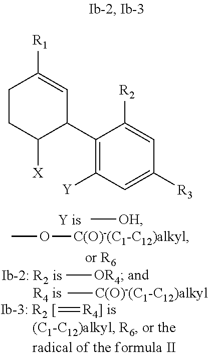

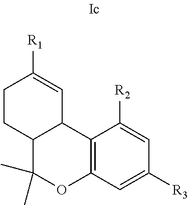

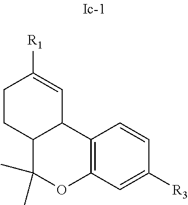

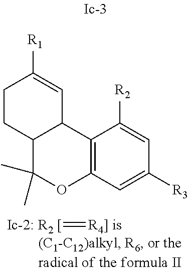

In further embodiments, the present invention provides a compound of the formula I according to any one of the embodiments above, wherein Y is —O— and together with X and the carbon atoms to which they are attached form a dihydropyran ring, i.e., a tetrahydrocannabinol (THC) derivative of the formula Ic in Table 2. Particular such compounds are those wherein (i) R₂ is H (formula Ic-1 in Table 2); or (ii) R₂ is R₄; and R₄ is (C₁-C₁₂)alkyl, e.g., (C₁-C₈)alkyl or (C₁-C₄)alkyl, R₆, or the radical of the formula II (formula Ic-2 in Table 2). More particular such compounds are those wherein R₁ is —CH₃, —CH₂F, —CH₂—OH, —CH₂—O—C(O)—(C₁-C₁₂)alkyl, e.g., —CH₂—O—C(O)—(C₁-C₈)alkyl or —CH₂—O—C(O)—(C₁-C₄)alkyl, or —CH₂—R₆; R₃ is R₅; and R₅ is (C₁-C₁₂)alkyl, (C₃-C₈)cycloalkylene-(C₁-C₁₂)alkyl, R₆, or the radical of the formula II.

Specific desoxy-CBD derivatives and conjugates thereof of the formula Ia are shown in Table 3, and are compounds of the formula I, wherein: (i) R₁ is —CH₃; R₂ is —OH; R₃ is R₅; and R₅ is 2-methyloctan-2-yl, 3-methyloctan-2-yl, 2-methylpentan-2-yl, 3-methylhexan-2-yl, 3-methylheptan-2-yl, 3-methylnonan-2-yl, octan-2-yl; 2-methylheptyl; 3-methyloct-2-en-2-yl, 2-pentylcyclopropyl, 2-pentylcyclobutyl, 1-methyl-2-pentylcyclopropyl, or the radical of the formula II (herein identified compound 101, 102, 103, 104, 105, 106, 107, 108, 109, 110, 111, 112 and 113, respectively); (ii) R₁ is —CH₂F; R₂ is —OH; R₃ is R₅; and R₅ is 3-methyloctan-2-yl (herein identified compound 114); (iii) R₁ is —CH₃; R₂ is R₄; R₃ is R₅; R₄ is R₆; R₅ is pentyl, 2-methyloctan-2-yl, 3-methyloctan-2-yl, or the radical of the formula II; and R₆ is naproxen linked through the carboxyl group thereof (herein identified compound 115, 116, 117 and 118, respectively); (iv) R₁ is —CH₂—OH; R₂ is —OH; R₃ is R₅; and R₅ is pentyl, 2-methyloctan-2-yl, 3-methylpentane-2-yl, 3-methyloctan-2-yl, or 2-methylbutan-2-yl (herein identified compound 119, 120, 121, 122 and 123, respectively); (v) R₁ is —CH₂—OH; R₂ is R₄; R₃ is R₅; R₄ is R₆; R₅ is pentyl, or 2-methyloctan-2-yl; and R₆ is naproxen linked through the carboxyl group thereof (herein identified compound 124 and 125, respectively); (vi) R₁ is —CH₂—R₆; R₂ is —OH; R₃ is R₅; R₅ is pentyl, 2-methyloctan-2-yl, 3-methyloctan-2-yl, or the radical of the formula II; and R₆ is betaine linked through the carboxyl group thereof (herein identified compound 126, 127, 128 and 129, respectively); (vii) R₁ is —CH₂—R₆; R₂ is —OH; R₃ is R₅; R₅ is pentyl; and R₆ is naproxen linked through the carboxyl group thereof (herein identified compound 130); (viii) R₁ is —CH₂—R₆ wherein R₆ is betaine linked through the carboxyl group thereof; R₂ is R₄; R₃ is R₅; R₄ is R₆ wherein R₆ is naproxen linked through the carboxyl group thereof; and R₅ is R₅ is pentyl, 2-methyloctan-2-yl, or 3-methyloctan-2-yl (herein identified compound 131, 132 and 133, respectively); or (ix) R₁ is —CH₂—R₆ wherein R₆ is naproxen linked through the carboxyl group thereof; R₂ is R₄; R₃ is R₅; R₄ is R₆ wherein R₆ is betaine linked through the carboxyl group thereof; and R₅ is pentyl, or 2-methyloctan-2-yl (herein identified compound 134 and 135, respectively).

TABLE 3
Specific desoxy-CBD derivatives and conjugates thereof of the formula Ia

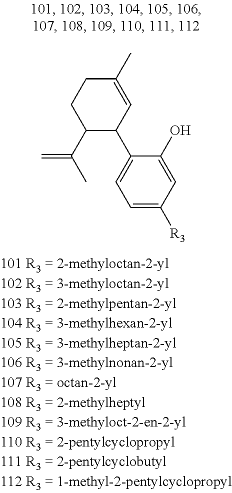

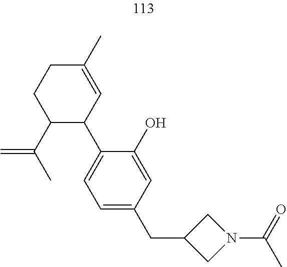 [SMILES: C=C(C)C1CCC(C)=CC1c1ccc(CC2CN(C(C)=O)C2)cc1O]

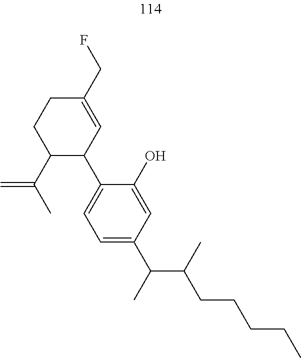 [SMILES: C=C(C)C1CCC(CF)=CC1c1ccc(C(C)C(C)CCCCC)cc1O]

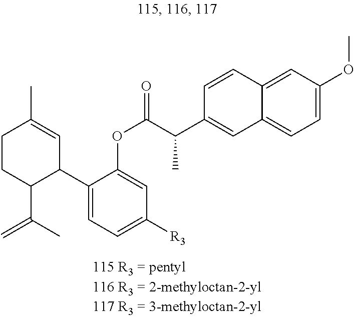

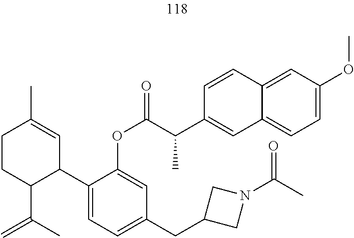 [SMILES: C=C(C)C1CCC(C)=CC1c1ccc(CC2CN(C(C)=O)C2)cc1OC(=O)[C@@H](C)c1ccc2cc(OC)ccc2c1]

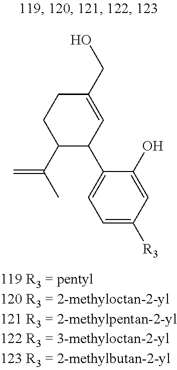

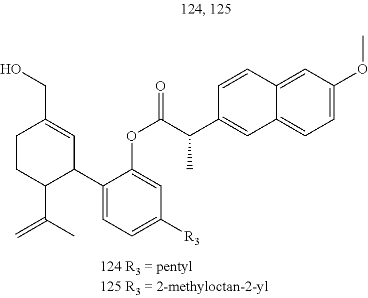

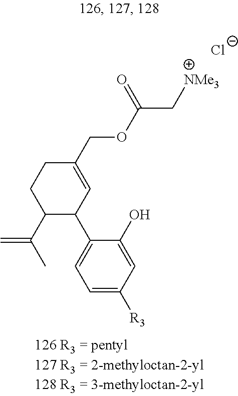

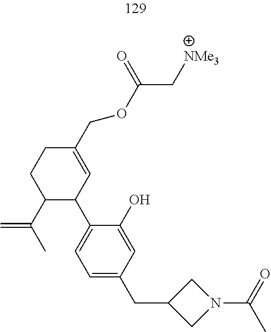

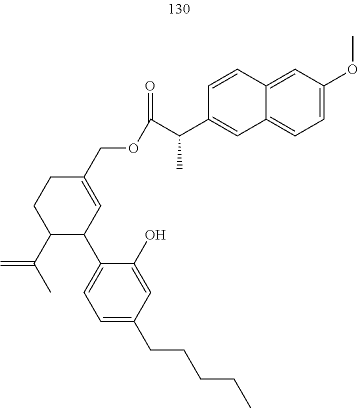 [SMILES: C=C(C)C1CCC(COC(=O)[C@@H](C)c2ccc3cc(OC)ccc3c2)=CC1c1ccc(CCCCC)cc1O]

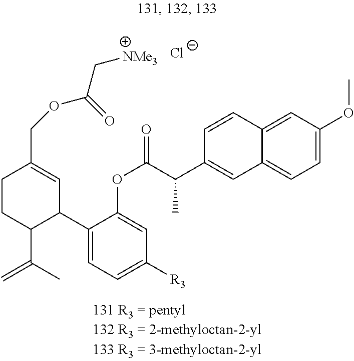

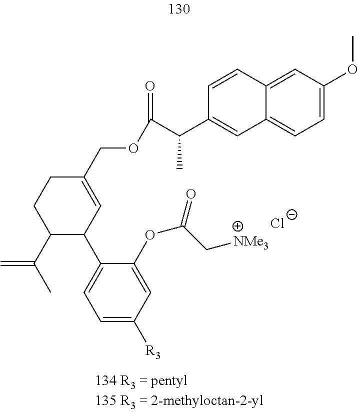

Specific conjugates of CBD derivatives of the formula Ib are shown in Table 4, and are compounds of the formula I, wherein: (i) Y is —OH; R₁ is CH₃; R₂ is —OH; R₃ is R₅; R₅ is R₆; and R₆ is veliparib or a derivative thereof, linked directly through the methyl group thereof (herein identified compound 136); (ii) Y is —OH; R₁ is —CH₃; R₂ is —OH; R₃ is R₅; R₅ is R₆; and R₆ is PJ34 linked through the dimethylamino group thereof and via a linker of the formula —CH₂—O—C(O)—(CH₂)ₙ—C(O)—O—, wherein n is an integer of 1-3 (herein identified compound 137); (iii) Y is —OH; R₁ is —CH₃; R₂ is —OH; R₃ is R₅; R₅ is R₆; and R₆ is niraparib linked through the nitrogen atom of the piperidine ring and via a linker of the formula —CH₂—O—C(O)—(CH₂)ₙ—C(O)—O—, wherein n is an integer of 1-3 (herein identified compound 138); (iv) Y is —OH; R₁ is —CH₂—R₆; R₂ is —OH; R₃ is R₅; R₅ is pentyl; and R₆ is codeine linked through the nitrogen atom thereof and via a linker of the formula —CH₂—O—C(O)—(CH₂)ₙ—C(O)—O—, wherein n is an integer of 1-3 (herein identified compound 139); (v) Y is —OH; R₁ is —CH₂—R₆; R₂ is —OH; R₃ is R₅; R₅ is pentyl; and R₆ is PJ34 linked through the dimethylamino group thereof and via a linker of the formula —CH₂—O—C(O)—(CH₂)ₙ—C(O)—O—, wherein n is an integer of 1-3 (herein identified compound 140); (vi) Y is —OH; R₁ is —CH₂—R₆; R₂ is —OH; R₃ is R₅; R₅ is pentyl; and R₆ is niraparib linked through the nitrogen atom of the piperidine ring and via a linker of the formula —CH₂—O—C(O)—(CH₂)ₙ—C(O)—O—, wherein n is an integer of 1-3 (herein identified compound 141); (vii) Y is —OH; R₁ is CH₃; R₂ is R₄; R₃ is R₅; R₄ is R₆; R₅ is pentyl; and R₆ is codeine linked through the nitrogen atom thereof and via a linker of the formula —CH₂—O—C(O)—(CH₂)ₙ—C(O)—O—, wherein n is an integer of 1-3 (herein identified compound 142); (viii) Y is —OH; R₁ is —CH₃; R₂ is R₄; R₃ is R₅; R₄ is R₆; R₅ is pentyl; and R₆ is PJ34 linked through the dimethylamino group thereof and via a linker of the formula —CH₂—O—C(O)—(CH₂)ₙ—C(O)—O—, wherein n is an integer of 1-3 (herein identified compound 143); (ix) Y is —OH; R₁ is —CH₃; R₂ is R₄; R₃ is R₅; R₄ is R₆; R₅ is pentyl; and R₆ is niraparib linked through the nitrogen atom of the piperidine ring and via a linker of the formula —CH₂—O—C(O)—(CH₂)ₙ—C(O)—O—, wherein n is an integer of 1-3 (herein identified compound 144); (x) Y is R₄ wherein R₄ is R₆ and R₆ is betaine linked through the carboxyl group thereof; R₁ is CH₃; R₂ is R₄; R₃ is R₅; R₄ is R₆ wherein R₆ is betaine linked through the carboxyl group thereof; and R₅ is R₆ wherein R₆ is veliparib or a derivative thereof, linked directly through the methyl group thereof (herein identified compound 145); or (xi) Y is R₄ wherein R₄ is R₆; R₁ is —CH₃; R₂ is R₄; R₃ is R₅; R₄ is R₆; R₅ is pentyl; and R₆ each independently is codeine linked through the nitrogen atom thereof and via a linker of the formula —CH₂—O—C(O)—(CH₂)ₙ—C(O)—O—, wherein n is an integer of 1-3 (herein identified compound 146).

TABLE 4
Specific conjugates of CBD derivatives of the formula Ib

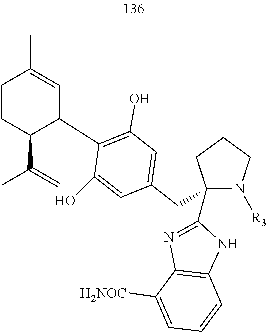

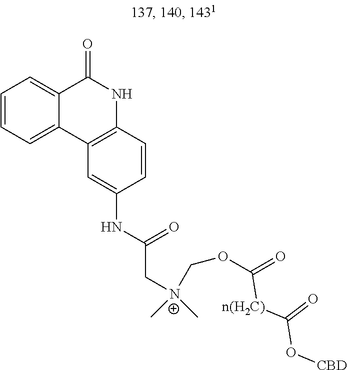

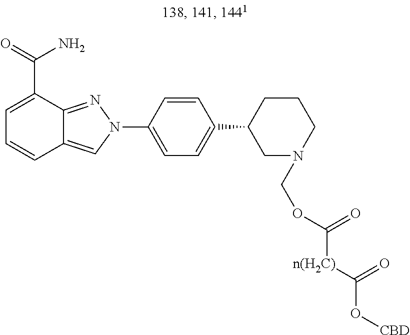

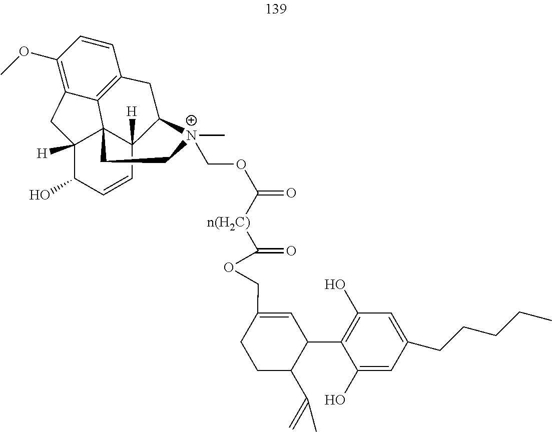

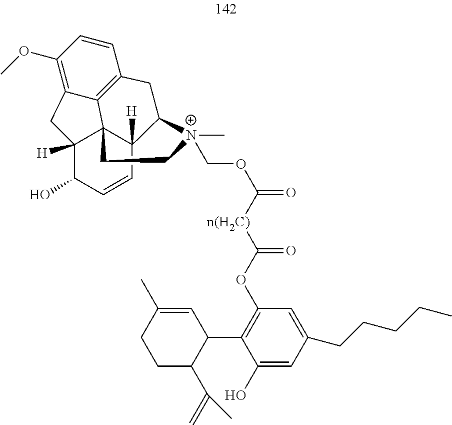

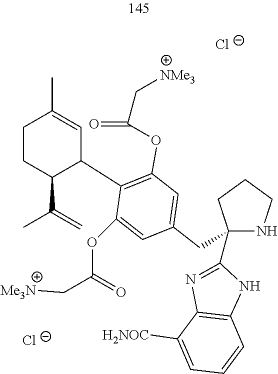

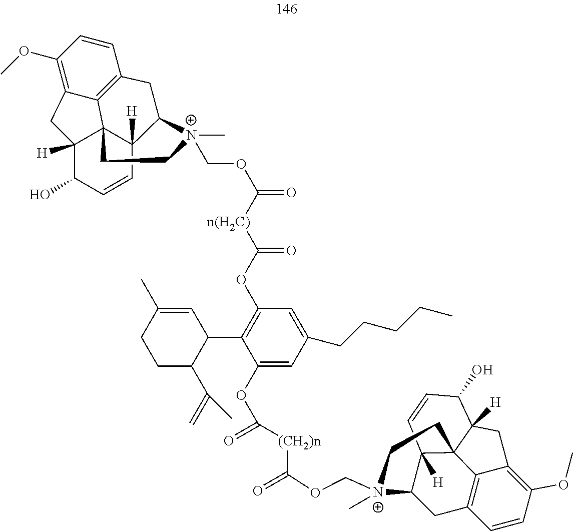

¹The drug is linked to the carbon atom at position 1 (compounds 143 and 144); 3 (compounds 137 and 138),
or 7 (compounds 140 and 141) in the compound of the formula I.

Specific THC derivatives and conjugates thereof of the formula Ic are shown in Table 5, and are compounds of the formula I, wherein: (i) R₁ is —CH₃; R₂ is H; R₃ is R₅; and R₅ is 3-methyloctan-2-yl, 2-methyloctan-2-yl, or 2-methylpentan-2-yl (herein identified compound 147, 148 and 149, respectively); (ii) R₁ is —CH₂—OH; R₂ is H; R₃ is R₅; and R₅ is pentyl, or 2-methylpentan-2-yl (herein identified compound 150 and 151, respectively); (iii) R₁ is —CH₃; R₂ is R₄; R₃ is R₅; R₄ is R₆; R₅ is propyl; and R₆ is naproxen linked through the carboxyl group thereof (herein identified compound 152); or (iv) R₁ is —CH₂—R₆ wherein R₆ is betaine linked through the carboxyl group thereof; R₂ is R₄; R₃ is R₅; R₄ is R₆ wherein R₆ is naproxen linked through the carboxyl group thereof; and R₅ is propyl (herein identified compound 153).

The compounds of the general formula I or III may have one or more asymmetric centers, and may accordingly exist both as enantiomers, i.e., optical isomers (R, S, or racemate, wherein a certain enantiomer may have an optical purity of 90%, 95%, 99% or more) and as diastereoisomers. Specifically, those chiral centers may be in the carbon atoms at positions 9 or 10 in the compound of the formula I; and in the carbon atom at position 2 of the 2H-chromene in the compound of the formula III (shown with an asterisk in the formula III). The present invention encompasses all such enantiomers, isomers and mixtures thereof, as well as pharmaceutically acceptable salts and solvates thereof.

TABLE 5
Specific THC derivatives and conjugates thereof of the formula Ic

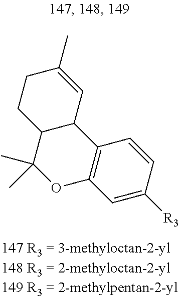

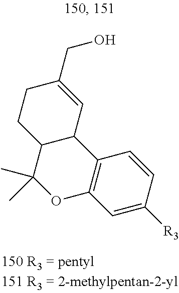

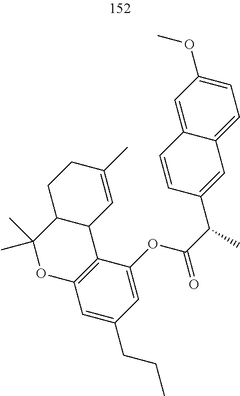 [SMILES: CCCc1cc(OC(=O)[C@@H](C)c2ccc3cc(OC)ccc3c2)c2c(c1)OC(C)(C)C1CCC(C)=CC21]

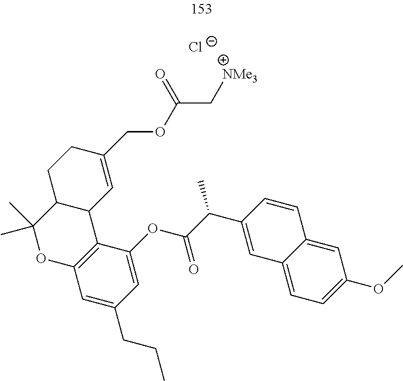

Optically active forms of the compounds of the general formula I and III may be prepared using any method known in the art, e.g., by resolution of the racemic form by recrystallization techniques; by chiral synthesis; by extraction with chiral solvents; or by chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

The cannabinoid compounds of formula I are CBD-, desoxy-CBD- or desoxy-THC-derivatives useful for neuroprotection, treating pain, or treating a disease associated with alpha-1 glycine receptor (α1GlyR) and/or alpha-3 glycine receptor (α3GlyR) deficiency. As shown in the experimental section herein, some of these compounds bind and activate the α1GlyR and/or α3GlyR in the CNS, and are thus capable of inhibiting nociceptive transmission and consequently exerting an analgesic effect. Some of these compounds, having one or more R₆ groups, are in fact conjugates wherein the cannabinoid molecule is conjugated to, e.g., an analgesic drug such as an NSAID or an opiate, in order to provide two complementary independent and non-overlapping analgesic effects. In those conjugates, the cannabinoid molecule and the analgesic drug are linked via an ester bond that is susceptible to hydrolysis by enzymes within the body, and it is therefore expected that administration of the conjugate will result in the separation of the two molecules in vivo.

A water solubilizing moiety, covalently conjugated via an ester bond to certain of the cannabinoid molecules, is expected to undergo rapid hydrolysis via esterases in bodily fluids, yielding the payload cannabinoid entity. The water solubilizing moiety is expected to allow for stable storage of the intact prodrug conjugate in aqueous solution, whereby it can be administered readily to a human via a blood vessel or a tissue. When administered enterally or rectally, it is expected that the hydrophilicity conferred by the water solubilizing moiety will reduce the intrinsic lipophilicity of the parent cannabinoid molecule, thereby facilitating gastrointestinal uptake.

A PARP inhibitor covalently conjugated via an ester bond to certain of the cannabinoid molecules is expected to undergo rapid hydrolysis via esterases in bodily fluids, yielding the payload cannabinoid entity and the PARP inhibitor. PARP inhibitors per se are expected to provide neuroprotection in the setting of a variety of neurological insults, including ischemia-reperfusion, stroke, hypoxia, anoxia, hyperammonemia, meningitis, encephalitis, traumatic brain injury, and spinal cord injury. The mechanism of action whereby PARP inhibitors confer this benefit is multifactorial and includes both anti-inflammatory actions as well as a preservation of intracellular pools of high energy phosphates and nucleotides. Both of these mechanisms block injury-mediated cell death (necrosis and apoptosis). The biological pathways invoked by administration of a PARP inhibitor differ from those triggered by administration of a cannabinoid, hence the combined administration of a PARP inhibitor and a cannabinoid (via a conjugated molecular prodrug) is anticipated to have a superior activity than the administration of merely one of the two components alone.

An iNOs inhibitor covalently conjugated via an ester bond to certain of the cannabinoid molecules is expected to undergo rapid hydrolysis via esterases in bodily fluids, yielding the payload cannabinoid entity and the iNOs inhibitor. iNOs inhibitors per se are expected to provide neuroprotection in the setting of a variety of neurological insults, including ischemia-reperfusion, stroke, hypoxia, anoxia, hyperammonemia, meningitis, encephalitis, traumatic brain injury, and spinal cord injury. The mechanism of action whereby iNOs inhibitors confer this benefit is multifactorial and includes both anti-inflammatory actions as well as a reduction in the levels of peroxynitrite, a highly toxic nitrosating and oxidizing species produced by the diffusion-limited reaction of nitric oxide and superoxide anion. The biological pathways invoked by administration of an iNOs inhibitor differ from those triggered by administration of a cannabinoid, hence the combined administration of an iNOs inhibitor and a cannabinoid (via a conjugated molecular prodrug) is anticipated to have a superior activity than the administration of merely one of the two components alone.

A cycloxygenase (COX-1 and COX2) inhibitor covalently conjugated via an ester bond to certain of the cannabinoid molecules is expected to undergo rapid hydrolysis via esterases in bodily fluids, yielding the payload cannabinoid entity and the COX inhibitor. COX inhibitors per se are expected to provide analgesia in the setting of a variety of painful settings, including inflammation, nerve compression, thermal injury, mechanical pressure, blunt trauma, penetrating trauma, and laceration or surgical incision. The mechanism of action whereby COX inhibitors confer this benefit is multifactorial and includes the suppression of anti-nociceptive pathways engendered by the activation of the α1GlyR and/or α3GlyR in the dorsal spinal cord. The biological pathways invoked by administration of a COX inhibitor differ from those triggered by administration of a cannabinoid, hence the combined administration of a COX inhibitor and a cannabinoid (via a conjugated molecular prodrug) is anticipated to have a superior activity than the administration of merely one of the two components alone. A particular COX inhibitor, naprosyn (also known as naproxen), blocks formation of prostaglandin E2 (PGE2), a bioactive lipid molecule that markedly inhibits ascending anti-nociceptive pathways triggered by activation of the α3GlyR and/or α1GlyR. The conjugation of naprosyn and the cannabinoid molecule is thus expected to produce an additive or synergetic analgesic effect because the two molecules, once separated from one another in the body, will be able to act in concert to activate discrete sections of a common anti-nociceptive signaling pathway.

An analgesic opiate covalently conjugated via an ester bond to certain of the cannabinoid molecules is expected to undergo rapid hydrolysis via esterases in bodily fluids, yielding the payload cannabinoid entity and the analgesic opiates. Opiates per se are expected to provide analgesia in the setting of a variety of painful settings, including inflammation, nerve compression, thermal injury, mechanical pressure, blunt trauma, penetrating trauma, and laceration or surgical incision. The mechanism of action whereby opiates confer this benefit is multifactorial and includes actions at multiple levels of the spinal cord and brain. The biological pathways invoked by administration of an opiate differ from those triggered by administration of a cannabinoid, hence the combined administration of an opiate and a cannabinoid (via a conjugated molecular prodrug) is anticipated to have a superior activity than the administration of merely one of the two components alone.

The type of cannabinoid optimally utilized for conferring analgesia or neuroprotection is related to its chemical structure, as it is known that various cannabinoids differ significantly in structure and biological activity. Preferred cannabinoids for conjugation into prodrugs according to the present invention are those that bind to various receptors involved in the pain response. These include ion channel pathways in the spinal cord and brain, especially those that bind to the α3GlyR and/or α1GlyR in the spinal cord. The use of desoxy-CBD is established to bind to the α3GlyR and/or α1GlyR, and exert analgesic effects via these receptors engagement. Other analgesic cannabinoids include THC, cannabichrome, tetrahydrocannabivarin (THCV), and CBD, as well as other cannabinoids that are present in a lower concentration in Cannabis satavis. It is expected that the hydrolysis of the prodrug conjugates from the cannabinoid moieties will free the payload cannabinoid of potential steric hindrance and thereby permit the cannabinoid molecules to reach and activate their intended biological target.

Additional changes to the cannabinoid payload are also intended to facilitate binding to and activation of the biological target and thus increase the potency of the analgesic or neuroprotective action. These structural alterations include changes to the alkane tail of the cannabinoid molecule, including alterations in chain length, structure, polarity, and lipophilicity.

The analgesic cannabinoid prodrugs formed from the conjugation of a cannabinoid with non-steroidal anti-inflammatory drugs, such as naprosyn, are not expected to depress respiratory drive and thus may prove advantageous in those medical settings where inducing respiratory depression is not desirable. The absence of respiratory depression of the conjugates contrasts with the undesirable side-effects of most opiate analgesics, many of which are well known to be associated with suppression of respiratory drive.

The analgesic cannabinoid prodrugs formed from the conjugation of a cannabinoid with opiates may permit lower doses of opiates to be employed effectively, thereby minimizing the opiate-induced depression of respiratory drive. This feature may prove advantageous in those medical settings where inducing respiratory depression is not desirable.

In a further aspect, the present invention thus provides a pharmaceutical composition comprising a cannabinoid compound of the formula I or III, each as defined in any one of the embodiments above, or an enantiomer, diastereomer, racemate, or pharmaceutically acceptable salt thereof, herein also identified as the active agent, and a pharmaceutically acceptable carrier. Particular such pharmaceutical compositions comprise, as the active agent, a compound of the formula Ia, lb or Ic selected from those specifically shown in Tables 3-5, or an enantiomer, diastereomer, racemate, or pharmaceutically acceptable salt thereof.

The pharmaceutical composition of the invention may be used for providing neuroprotection, treating pain, or treating a disease associated with GlyR deficiency such as hyperekplexia disease.

In certain embodiments, the pharmaceutical composition of the invention is used for providing neuroprotection. Neuroprotection may be directed to the treatment of, e.g., stroke, ischemia-reperfusion injury of the brain or spinal cord, hypoxia, anoxia, meningitis, encephalitis, brain or spinal cord trauma, a neurodegenerative disease such as Alzheimer's disease, Huntington's disease, Parkinson's disease, amyotrophiclateralsclerosis, spinal muscular atrophy, and multiple sclerosis).

In other embodiments, the pharmaceutical composition of the invention is used for treating pain. Analgesia may be directed to the treatment of painful conditions induced by, e.g., thermal exposure, penetrating or blunt trauma, nerve compression, toxins and irritants, cancer, childbirth, vascular dilation, ischemia, infarction, laceration, inflammation, decompression sickness, fractures, dislocations of joints, obstruction of flow, mechanical pressure, surgery, post-operative conditions, and medical procedures.

In further embodiments, the pharmaceutical composition of the invention is used for treating a disease associated with GlyR deficiency, e.g., hyperekplexia disease.

The pharmaceutical compositions of the present invention can be provided in a variety of formulations, e.g., in a pharmaceutically acceptable form and/or in a salt form, as well as in a variety of dosages.

In one embodiment, the pharmaceutical composition of the present invention comprises a non-toxic pharmaceutically acceptable salt of a compound of the general formula I. Suitable pharmaceutically acceptable salts include acid addition salts such as, without being limited to, the mesylate salt, the maleate salt, the fumarate salt, the tartrate salt, the hydrochloride salt, the hydrobromide salt, the esylate salt, the p-toluenesulfonate salt, the benzenesulfonate salt, the benzoate salt, the acetate salt, the phosphate salt, the sulfate salt, the citrate salt, the carbonate salt, and the succinate salt. Additional pharmaceutically acceptable salts include salts of ammonium (NH₄⁺) or an organic cation derived from an amine of the formula R₄N⁺, wherein each one of the R₅ independently is selected from H, C₁-C₂₂, preferably C₁-C₆ alkyl, such as methyl, ethyl, propyl, isopropyl, n-butyl, sec-butyl, isobutyl, tert-butyl, n-pentyl, 2,2-dimethylpropyl, n-hexyl, and the like, phenyl, or heteroaryl such as pyridyl, imidazolyl, pyrimidinyl, and the like, or two of the R₅ together with the nitrogen atom to which they are attached form a 3-7 membered ring optionally containing a further heteroatom selected from N, S and O, such as pyrrolydine, piperidine and morpholine. Furthermore, where the compounds of the general formula I carry an acidic moiety, suitable pharmaceutically acceptable salts thereof may include metal salts such as alkali metal salts, e.g., lithium, sodium or potassium salts, and alkaline earth metal salts, e.g., calcium or magnesium salts.

Further pharmaceutically acceptable salts include salts of a cationic lipid or a mixture of cationic lipids. Cationic lipids are often mixed with neutral lipids prior to use as delivery agents. Neutral lipids include, but are not limited to, lecithins; phosphatidylethanolamine; diacyl phosphatidylethanolamines such as dioleoyl phosphatidylethanolamine, dipalmitoyl phosphatidylethanolamine, palmitoyloleoyl phosphatidylethanolamine and distearoyl phosphatidylethanolamine; phosphatidylcholine; diacyl phosphatidylcholines such as dioleoyl phosphatidylcholine, dipalmitoyl phosphatidylcholine, palmitoyloleoyl phosphatidylcholine and distearoyl phosphatidylcholine; phosphatidylglycerol; diacyl phosphatidylglycerols such as dioleoyl phosphatidylglycerol, dipalmitoyl phosphatidylglycerol and distearoyl phosphatidylglycerol; phosphatidylserine; diacyl phosphatidylserines such as dioleoyl- or dipalmitoyl phosphatidylserine; and diphosphatidylglycerols; fatty acid esters; glycerol esters; sphingolipids; cardiolipin; cerebrosides; ceramides; and mixtures thereof. Neutral lipids also include cholesterol and other 3β hydroxy-sterols.

Examples of cationic lipid compounds include, without being limited to, Lipofectin® (Life Technologies, Burlington, Ontario) (1:1 (w/w) formulation of the cationic lipid N-[1-(2,3-dioleyloxy)propyl]-N,N,N-trimethylammonium chloride and dioleoylphosphatidyl-ethanolamine); Lipofectamine™ (Life Technologies, Burlington, Ontario) (3:1 (w/w) formulation of polycationic lipid 2,3-dioleyloxy-N-[2(spermine-carboxamido)ethyl]-N,N-dimethyl-1-propanamin-iumtrifluoroacetate and dioleoylphosphatidyl-ethanolamine), Lipofectamine Plus (Life Technologies, Burlington, Ontario) (Lipofectamine and Plus reagent), Lipofectamine 2000 (Life Technologies, Burlington, Ontario) (Cationic lipid), Effectene (Qiagen, Mississauga, Ontario) (Non liposomal lipid formulation), Metafectene (Biontex, Munich, Germany) (Polycationic lipid), Eu-fectins (Promega Biosciences, San Luis Obispo, Calif.) (ethanolic cationic lipids numbers 1 through 12: C₅₂H₁₀₆N₆O₄.4 CF₃CO₂H, C₈₈H₁₇₈N₈O₄.S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂O₆N₁₂O₄S₂.8CF₃CO₂H, C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, C₄₁H₇₈NO₈P); Cytofectene (Bio-Rad, Hercules, Calif.) (mixture of a cationic lipid and a neutral lipid), GenePORTER® (Gene Therapy Systems, San Diego, Calif.) (formulation of a neutral lipid (Dope) and a cationic lipid) and FuGENE 6 (Roche Molecular Biochemicals, Indianapolis, Ind.) (Multi-component lipid based non-liposomal reagent).

The pharmaceutically acceptable salts of the present invention may be formed by conventional means, e.g., by reacting a free base form of the active agent, i.e., the compound of the general formula I or III, with one or more equivalents of the appropriate acid in a solvent or medium in which the salt is insoluble, or in a solvent such as water which is removed in vacuo or by freeze drying, or by exchanging the anion/cation of an existing salt for another anion/cation on a suitable ion exchange resin.

The pharmaceutical compositions provided by the present invention may be prepared by conventional techniques, e.g., as described in Remington: The Science and Practice of Pharmacy, 19^{th} Ed., 1995. The compositions can be prepared, e.g., by uniformly and intimately bringing the active agent into association with a liquid carrier, a finely divided solid carrier, or both, and then, if necessary, shaping the product into the desired formulation. The compositions may be in liquid, solid or semisolid form and may further include pharmaceutically acceptable fillers, carriers, diluents or adjuvants, and other inert ingredients and excipients. In one embodiment, the pharmaceutical composition of the present invention is formulated as nanoparticles.

The compositions can be formulated for any suitable route of administration, but they are preferably formulated for parenteral, e.g., intravenous, intraarterial, intramuscular, intraperitoneal, intrathecal, intrapleural, intratracheal, subcutaneous, or topical administration, as well as for inhalation. The dosage will depend on the state of the patient and will be determined as deemed appropriate by the practitioner.

The pharmaceutical composition of the invention may be in the form of a sterile injectable aqueous or oleagenous suspension, which may be formulated according to the known art using suitable dispersing, wetting or suspending agents. The sterile injectable preparation may also be a sterile injectable solution or suspension in a non-toxic parenterally acceptable diluent or solvent. Acceptable vehicles and solvents that may be employed include, without limiting, water, Ringer's solution, polyethylene glycol (PEG), 2-hydroxypropyl-β-cyclodextrin (HPCD), Tween-80, and isotonic sodium chloride solution.

Pharmaceutical compositions according to the present invention, when formulated for inhalation, may be administered utilizing any suitable device known in the art, such as metered dose inhalers, liquid nebulizers, dry powder inhalers, sprayers, thermal vaporizers, electrohydrodynamic aerosolizers, and the like.

Pharmaceutical compositions according to the present invention, when formulated for administration route other than parenteral administration, may be in a form suitable for oral use, e.g., as tablets, troches, lozenges, aqueous, or oily suspensions, dispersible powders or granules, emulsions, hard or soft capsules, or syrups or elixirs.

Pharmaceutical compositions intended for oral administration might be formulated so as to inhibit the release of the active agent in the stomach, i.e., delay the release of the active agent until at least a portion of the dosage form has traversed the stomach, in order to avoid the acidity of the gastric contents from hydrolyzing the active agent. Particular such compositions are those wherein the active agent is coated by a pH-dependent enteric-coating polymer. Examples of pH-dependent enteric-coating polymer include, without being limited to, Eudragit® S (poly(methacrylicacid, methylmethacrylate), 1:2), Eudragit® L 55 (poly(methacrylicacid, ethylacrylate), 1:1), Kollicoat® (poly(methacrylicacid, ethylacrylate), 1:1), hydroxypropyl methylcellulose phthalate (HPMCP), alginates, carboxymethylcellulose, and combinations thereof. The pH-dependent enteric-coating polymer may be present in the composition in an amount from about 10% to about 95% by weight of the entire composition.

Pharmaceutical compositions intended for oral administration may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

The pharmaceutical compositions of the invention may be formulated for controlled release of the active agent. Such compositions may be formulated as controlled-release matrix, e.g., as controlled-release matrix tablets in which the release of a soluble active agent is controlled by having the active diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other configurations, the compositions comprise the active agent formulated for controlled release in microencapsulated dosage form, in which small droplets of the active agent are surrounded by a coating or a membrane to form particles in the range of a few micrometers to a few millimeters.

Another contemplated formulation is depot systems, based on biodegradable polymers, wherein as the polymer degrades, the active ingredient is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

In yet a further aspect, the present invention relates to a cannabinoid compound of the formula I or III, each as defined in any one of the embodiments above, or an enantiomer, diastereomer, racemate, or pharmaceutically acceptable salt thereof, for use in providing neuroprotection, treating pain, or treating a disease associated with GlyR deficiency such as hyperekplexia disease.

In still a further aspect, the present invention relates to use of a cannabinoid compound of the formula I or III, each as defined in any one of the embodiments above, or an enantiomer, diastereomer, racemate, or pharmaceutically acceptable salt thereof, for the preparation of a pharmaceutical composition for providing neuroprotection, treating pain, or treating a disease associated with GlyR deficiency such as hyperekplexia disease.

In another aspect, the present invention relates to a method for providing neuroprotection, treating pain, or treating a disease associated with GlyR deficiency such as hyperekplexia diseas, in an individual in need thereof, comprising administering to said individual an effective amount of a cannabinoid compound of the formula I or III, each as defined in any one of the embodiments above, or an enantiomer, diastereomer, racemate, or pharmaceutically acceptable salt thereof.

The invention will now be illustrated by the following non-limiting Examples.

### EXAMPLES

### Experimental

General Procedure for Coupling Reaction of Desoxy-Olivetol Derivates with Dementhene

To a three naked flask occupied with thermometer and anhydrous MgSO₄ was added desoxy-olivetol in dry DCM. The reaction mixture was cooled to −10 and 0.01 equiv. of BF₃OEt₂ was added. Dementhene in dry DCM (1.2 equiv) was added to reaction mixture dropwise. The reaction monitored by TLC, stirred for 4-5 hours, quenched with sat. bicarbonate and extracted with DCM. The crude was purified by silica gel chromatography (10% Et₂O in hexane).

General Procedure for Coupling Reaction of Desoxy-Olivetol Derivates with 7-OH Dementhene

To a three naked flask occupied with thermometer and anhydrous MgSO₄ was added desoxy-olivetol in dry DCM. The reaction mixture was cooled to −10 and 0.01 equiv. of BF₃OEt₂ was added. 7-OAc-dementhene (CAS: 936001-98-8) in dry DCM (1.2 equiv) was added to reaction mixture dropwise. The reaction monitored by TLC, stirred for 4-5 hours, quenched with sat. bicarbonate and extracted with DCM. The crude was purified by silica gel chromatography (10% Et₂O in hexane). Hydrolysis of acetate protecting group was preform by addition 1 N NaOH (1.2 equiv.) to the coupling product in EtOH. The reaction stirred at room temperature a few hours and monitored by TLC. The reaction neutralized with sat. NH₄Cl, evaporated and extracted with DCM/brine. The organic phases dried over Na₂SO₄ filtrated and evaporated.

### Synthesis of 2-(3-methoxyphenyl)-2-butanone

As depicted in Scheme 1, the intermediate 2-(3-methoxyphenyl)-2-butanone was prepared from 3-methoxy acetophenone. 3-Methoxy-acetophenone was treated with methoxymethyl triphenyl phosphonium salt, and upon hydrolysis, Grignard reaction and oxidation provided 2-(3-methoxyphenyl)-2-butanone.

### Example 1. Synthesis of Desoxy-CBD

To a suspension of magnesium sulfate and 3-pentylphenol (1 eq) in DCM was added catalytic amounts of BF₃-Et₂O at −10° C. and a solution of cis-p-mentha-2,8-dien-1-ol in DCM was added slowly over 10 min. The reaction was stirred for 1.5 hr at −10° C. and quenched with saturated sodium bicarbonate solution. The reaction mixture was extracted with DCM, dried on dry sodium sulphate and concentrated. The crude product was purified by flash chromatography on silica gel (Et₂O/Hex) to afford the product. GC-MS: 298.3.

### Example 2. Synthesis of Desoxy-THC

To a suspension of magnesium sulfate and desoxy-CBD (1 eq) in DCM was added BF₃-Et₂O (1.1 eq) at −10° C. slowly. The reaction was stirred for 1.5 hr at −10° C. and quenched with saturated sodium bicarbonate. Compound was extracted with DCM, dried and evaporated. The crude product was purified by flash chromatography on silica gel (Et₂O/Hex) to afford the product. GC-MS: 298.

### Example 3. Synthesis of 7-OH-Desoxy-CBD (119)

To a suspension of magnesium sulfate and 3-pentylphenol (1 eq) in DCM was added catalytic amounts of BF₃-Et₂O at −10° C. and a solution of 7-acetoxy analog of cis-p-mentha-2,8-dien-1-ol in DCM was added slowly. The reaction was stirred for 1.5-3 hr at −10° C. and quenched with saturated sodium bicarbonate. It was extracted with DCM, dried and evaporated. The crude product was purified by flash chromatography on silica gel (Et₂O/Hex) to afford the acetate product. The acetate group was removed suing ethanol and 1M sodium hydroxide (1 eq) at 0° C. The reaction was stirred overnight, the ethanol was concentrated, and the residue was extracted in ethyl acetate. Ethyl acetate was dried on sodium sulphate and evaporated. The crude product was purified by flash chromatography on silica gel (Et₂O/Hex) to afford the desired product. GC-MS: 297 (loss of OH group).

### Example 4. Synthesis of Desoxy-CBD-Naproxene Prodrug

Naproxene (1.2 eq) was dissolved in ACN and DCC (1.2 eq) and catalytic amount of DMAP was added at 5° C. The suspension was stirred for 10 minutes and a solution of desoxy-CBD (1 eq) in ACN was added to it slowly. The reaction was kept overnight at room temperature. The reaction was complete. It was filtered and purified by flash chromatography on silica gel (Et₂O/Hex) to afford the product.

### Example 5. Synthesis of 1,2-dimethylalkyl-desoxy CBD

As depicted in Scheme 2, 2-(3-methoxyphenyl)-2-butanone was treated with various Wittig salt with C₂ to C₆ chain and after hydrolysis and demethylation reaction provided C₄ to C₈ 1,2-dimethyl-desoxyolivetol derivatives. The reaction of desoxy-olivetol derivatives and demethane in BF₃ etherate gave the corresponding 1,2-dimethylalkyl desoxy-CBD, i.e., 1,2-dimethylbutyl-, 1,2-dimethylpentyl-, 1,2-dimethylhexyl-, 1,2-dimethylheptyl-, or 1,2-dimethyloctyl-desoxy CBD.

1,2-dimethylheptyl-desoxy CBD (102). ¹H NMR (400 MHz, CDCl₃) δ 6.86 (d, J=7.1 Hz, 1H), 6.62 (s, 2H), 5.50 (d, J=20.8 Hz, 2H), 4.67 (s, 1H), 4.55 (s, 1H), 3.39 (d, J=8.6 Hz, 1H), 2.67-2.50 (m, 1H), 2.03-2.32 (m, 4H), 1.77, (s, 3H), 1.74-1.71 (m, 2H), 1.61-1.64 (m, 2H), 1.56 (S, 3H) 1.48-1.52 (m, 2H), 1.40-1.09 (m, 16H), 0.86 (t, J=6.8 Hz, 3H). m/z 354.4.

### Example 6. Synthesis of 1,1-dimethylheptyl- and 1,1-dimethylbutyl desoxy CBD, and 1,1-dimethylheptyl-desoxy THC

As depicted in Schemes 3-4, 1,1-dimethylheptyl desoxy CBD (101) and 1,1-dimethylbutyl desoxy CBD (103) were synthesized from 3-methoxy benzylnitrile. Methylation of bezonitrile using NaH and Mel in THF provided the dimethyl analog. The reduction of the cyano group with DIBAL gave the corresponding aldehyde. The Wittig reaction with C₅ and C₂ carbon chain Wittig salts provided the olefins. The hydrogenation, demethylation and coupling reaction with dementhane produced 101 and BPL-1841, respectively.

1,1-DMH desoxy CBD (101). ¹H NMR (400 MHz, CDCl₃) δ 6.87 (d, J=8.4 Hz, 1H), 6.79-6.71 (m, 2H), 5.54 (s, 1H), 5.44 (s, 1H), 4.69-4.62 (m, 1H), 4.55 (s, 1H), 3.43-3.29 (m, 1H), 2.38-2.13 (m, J=1.9 Hz, 2H), 2.13-1.99 (m, 1H), 1.86-1.67 (m, 5H), 1.57-1.48 (m, 6H), 1.30-1.12 (m, 13H), 1.06-0.95 (m, 2H), 0.83 (t, J=6.9 Hz, 3H). m/z 354.2.

1,1-Dimethylbutyl desoxy CBD (103). ¹H NMR (400 MHz, CDCl₃) δ 6.92-6.82 (m, 1H), 6.80-6.67 (m, 2H), 5.53 (s, 1H), 5.43 (s, 1H), 4.67 (s, 1H), 4.55 (s, 1H), 3.38 (d, J=8.3 Hz, 1H), 2.33-2.25 (m, 1H), 2.24-2.15 (m, 1H), 2.11-2.01 (m, 1H), 1.77 (s, 3H), 1.77-1.66 (m, 2H), 1.56 (d, J=2.6 Hz, 4H), 1.54-1.49 (m, 2H), 1.24 (s, 6H), 1.11-0.97 (m, 3H), 0.79 (t, J=7.3 Hz, 3H).

Cyclization of 1,1-DMH desoxy CBD in acid such as p-TSA or BF₃ provided its THC analog. ¹H NMR (400 MHz, CDCl₃) δ 7.22 (dd, J=8.1, 0.7 Hz, 1H), 6.85 (dd, J=8.1, 2.0 Hz, 1H), 6.76 (t, J=2.1 Hz, 1H), 5.95 (s, 1H), 3.17 (d, J=11.4 Hz, 1H), 2.11 (d, J=6.0 Hz, 2H), 1.89 (m, 1H), 1.73 (d, J=0.7 Hz, 3H), 1.56 (s, 3H), 1.54-1.51 (m, 2H), 1.44 (s, 3H), 1.41-1.36 (m, 1H), 1.24 (s, 6H), 1.18 (s, 6H), 1.11-1.01 (m, 3H), 0.84 (t, J=6.9 Hz, 3H). m/z 354.

### Example 7. Synthesis of 2-pentylcyclobutyl and 2-pentylcyclopropyl desoxy CBD

As depicted in Scheme 5, 2-(3-methoxyphenyl cyclobutanone was prepared from 3-methoxybenzaldehyde. The Wittig reaction, hydrogenation and coupling reaction with dementhane provided 2-pentylcyclobutyl desoxy CBD (111).

The cyclopropyl analog 110 was prepared from 3-tert-butyldimethylsilyl oxybenzaldehyde, as shown in Scheme 6, by performing Wittig reaction, cyclopropynation, silyl deprotection and coupling reaction with dementhane.

### Example 8. Synthesis of 2-methylheptyl- and 1-methylheptyl desoxy CBD

The synthesis of 2-methylheptyl desoxy CBD (BPL-1872) was performed as shown in Scheme 7. Wittig reaction, hydrogenation and demethylation reactions starting from 3-methoxybenzaldehyde gave access to the intermediate that can be coupled with dementhane in order to make 107. Synthesis of 1-methylheptyl desoxy-CBD (108) is displayed in Scheme 8.

2-Methylheptyl desoxy CBD (107). ¹H NMR (400 MHz, CDCl₃) δ 6.85 (d, J=8.1 Hz, 1H), 6.59 (d, J=4.9 Hz, 2H), 5.53 (s, 1H), 5.42 (s, 1H), 4.66 (s, 1H), 4.54 (s, 1H), 3.38 (d, J=8.5 Hz, 1H), 2.60-2.47 (m, 1H), 2.37-2.15 (m, 3H), 2.00-2.09 (m, 1H), 1.77 (s, 3H), 1.75-1.63 (m, 2H), 1.56 (s, 2H), 1.55 (s, 2H), 1.29 (ddd, J=26.6, 13.9, 6.6 Hz, 9H), 1.14-1.06 (m, 1H), 0.87 (t, J=7.0 Hz, 3H), 0.81 (d, J=6.6 Hz, 3H). m/z=340.4.

### Example 9. Synthesis of 1,2-dimethyl-1-heptenyl-desoxy CBD

The synthesis of 1,2-dimethyl-1-heptenyl desoxy CBD (109) was performed as depicted in Scheme 9. 3-Methoxy-acetophenone was treated with Wittig salt that was prepared from 2-bromoheptane. Demethylation reaction was carried out using BBr3. The coupling reaction with dementhane provided 109.

### Example 10. Synthesis of 7-OH-1,1-dimethylalkyl-desoxy CBD

The synthesis of 7-hydroxy-1,1-dimethylalkyl- and 7-hydroxy-1,2-dimethylalkyl desoxy CBD analogs was carried out as depicted in Schemes 4, 10 and 11. The 7-acetoxy,1-hydroxy dementhane or 1,7-dihydroxy dementhane were synthesized from limonene (Scheme 10) and then reacted with various 3-alkyl substituted phenols (Scheme 11). Thus, 3-(1,2-dimethylheptyl)-phenol on treatment with 7-acetoxy-dementhane produced 7-hydroxy-1,2-dimethylheptyl desoxy CBD (Scheme 10). The 7-fluoro-1,2-dimethylheptyl desoxy CBD was then prepared from 7-hydroxy-1,2-dimethylheptyl desoxy CBD using DAST.

7-OH-1,1-DMH-desoxy CBD (120). ¹H NMR (400 MHz, CDCl₃) δ 6.91 (d, J=8.0 Hz, 1H), 6.78 (d, J=8.1 Hz, 1H), 6.74 (s, 1H), 5.74 (s, 1H), 4.69 (s, 1H), 4.59 (s, 1H), 4.10 (s, 2H), 3.54 (d, J=8.7 Hz, 1H), 2.31 (dd, J=26.8, 17.3 Hz, 2H), 2.20 (s, 2H), 1.85 (s, 1H), 1.76 (dt, J=22.0, 9.6 Hz, 1H), 1.59 (s, 3H), 1.51 (dd, J=10.3, 6.3 Hz, 2H), 1.23 (bs, 8H), 1.17 (bs, 5H), 1.00 (s, 2H), 0.83 (t, J=6.8 Hz, 3H). m/z=370.

7-OH-1,1-dimethylbutyl-desoxy CBD (121). ¹H NMR (400 MHz, CDCl₃) δ 6.91 (d, J=8.0 Hz, 1H), 6.79 (dd, J=8.0, 1.7 Hz, 1H), 6.74 (d, J=1.7 Hz, 1H), 5.74 (s, 1H), 5.13 (s, 1H), 4.70 (s, 1H), 4.60 (s, 1H), 4.10 (s, 2H), 3.54 (d, J=7.9 Hz, 1H), 2.32 (td, J=11.9, 5.9 Hz, 1H), 2.20 (d, J=2.8 Hz, 2H), 1.86 (m, 1H), 1.82-1.69 (m, 2H), 1.60 (s, 3H), 1.57-1.43 (m, 2H), 1.24 (s, 6H), 1.04 (d, J=8.5 Hz, 2H), 0.80 (t, J=7.3 Hz, 3H).

### Example 11. Patch Clamp Electrophysiology

Currents were recorded by whole-cell patch clamp on HEK293 cells that stably expressed either the human α1 or α3 GlyRs. Cells were cultured on glass coverslips and placed into the recording chamber which was perfused by the standard extracellular solution containing (in mM): 140 NaCl, 5 KCl, 2 CaCl₂), 1 MgCl₂, 10 HEPES/NaOH and 10 glucose (pH 7.4 adjusted with NaOH). For HEK293 cell recordings we employed an intracellular solution consisting of (mM): 145 CsCl, 2 CaCl₂), 2 MgCl₂, 10 HEPES and 10 EGTA (pH 7.4 adjusted with CsOH; osmolarity 290 mOsm). HEK293 cell recordings were performed at a holding potential of −40 mV. Solutions were applied to cells via gravity induced perfusion via parallel microtubules under micromanipulator control with a solution exchange time of <250 ms. Experiments were conducted at room temperature (20-22° C.). Due to the irreversible nature of the drug actions, only one cell was tested per coverslip.

Patch pipettes were fabricated from borosilicate hematocrit tubing (Hirschmann Laborgerate, Eberstadt, Germany) and heat polished. Pipettes had tip resistances of 1-2 MW. Membrane currents were recorded using an Axopatch 200B amplifier and a Digidata 1440 analog-to-digital converter under control of pClamp10 software (Molecular Devices). Currents were filtered at 500 Hz and digitized at 2 KHz.

### Example 12. Experimental Protocol

After a stable whole cell recording was obtained, EC2 and saturating glycine concentrations activated currents with the anticipated magnitudes were verified. At al and α3 GlyRs, EC2 current magnitudes generally required glycine concentrations of 1 and 80 μM, respectively. The saturating concentration was invariably 2 mM. The standard experimental protocol involved EC2 glycine application for −3 s every 1 minute. When glycine was not being applied, the cells were continually and directly exposed to flowing drug solution. After 5 minutes of alternate applications of drug and EC2 glycine, 2 mM glycine was briefly applied every 2 minutes. Regular applications of saturating glycine after the 5 minute time point tended to enhance the drug-induced current magnitude effect further.

### Example 13. Cannabinoid Derivatives Modulate Al GlyR and α3 GlyR

The effects of cannabinoid derivatives on al GlyR and α3 GlyR-regulated ion channel currents, as assessed by the patch clamp method, are indicated in Table 6. In most cases, the effects of the derivatives on al GlyR regulation differ from those on α3 GlyR regulation, and especially in the case of 1,2-DMH desoxy CBD that is highly selective towards α3 GlyR.

TABLE 6
Modulation of α1 GlyR and α3 GlyR-
regulated ion channel currents

| | | α1 GlyR | A3 GlyR |
|---|---|---|---|
| Com- | | (fold | (fold |
| pound | | increase) | increase) |
| | desoxy-THC | 11.2 | 4.7 |
| 148 | 1,1-DMH-desoxy THC | 4.2 | 4 |
| | desoxy-CBD | 26.7 | 4.6 |
| 101 | 1,1-DMH-desoxy-CBD | 13 | 5 |
| 102 | 1,2-DMH-desoxy-CBD | no effect | 18 |
| 103 | 1,1-dimethylbutyl-desoxy-CBD | 4 | 10 |
| 119 | 7-hydroxy-desoxy-CBD | no effect | complete |
| | | | block |
| 121 | 7-hydroxy-1,1-dimethylbutyl-desoxy- | weak | weak |
| | CBD | inhibitory | inhibitory |
| | | effect | effect |
| 107 | 1′-monomethylheptyl-desoxy-CBD | nt | nt |
| 106 | 1,2-dimethyoctyl-desoxy-CBD | nt | nt |
| 109 | 1,2-dimethyl-1-heptenyl-desoxy-CBD | nt | nt |
| 104 | 1,2-dimethylpentyl-desoxy-CBD | nt | nt |
| 110 | 2-pentylcyclopropyl-desoxy-CBD | nt | nt |
| 111 | 2-pentylcyclobutyl-desoxy-CBD | nt | nt |
| 114 | 7-F,1,2-dimethylheptyl-desoxy-CBD | nt | nt |

* nt—not tested.

### APPENDIX

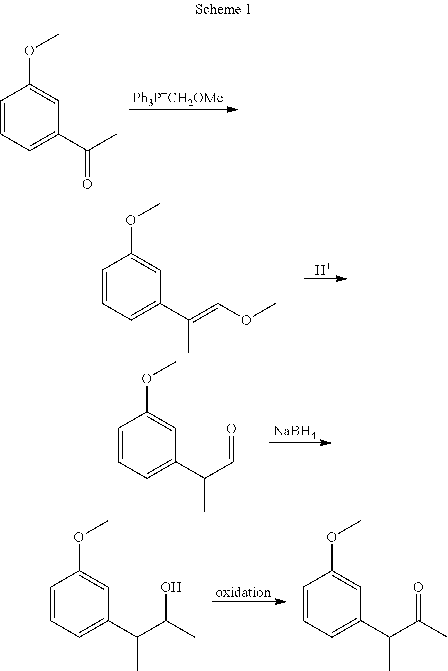

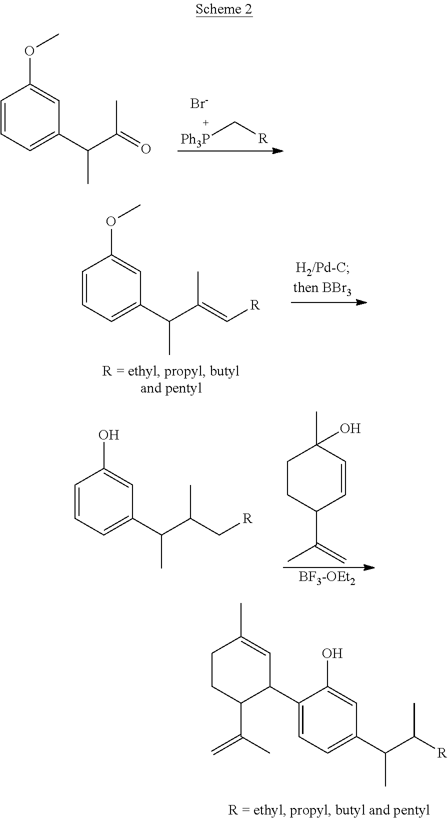

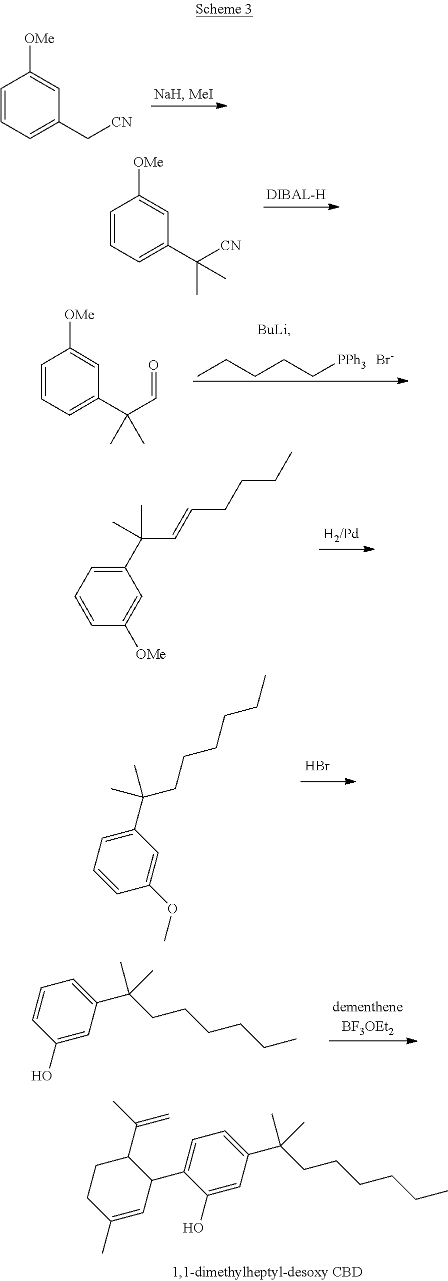

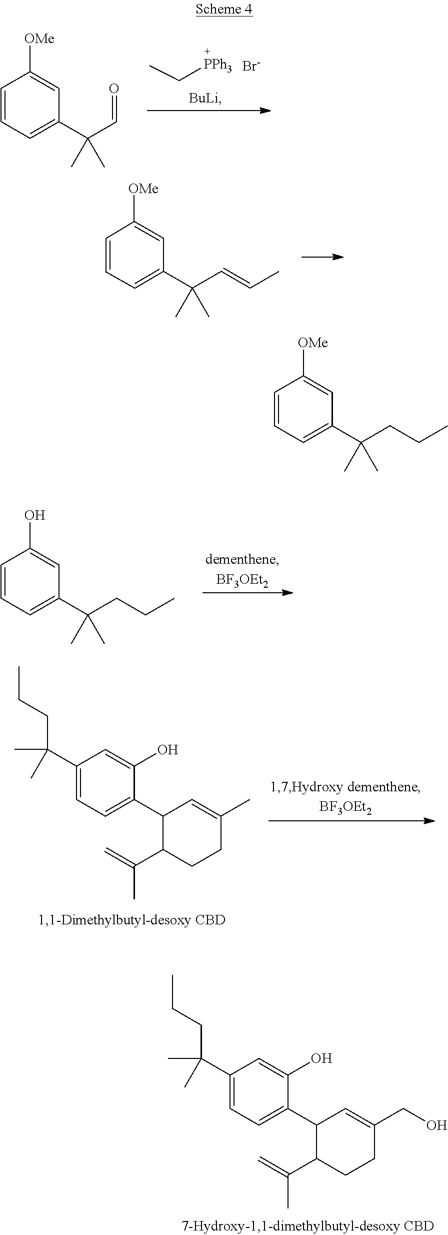

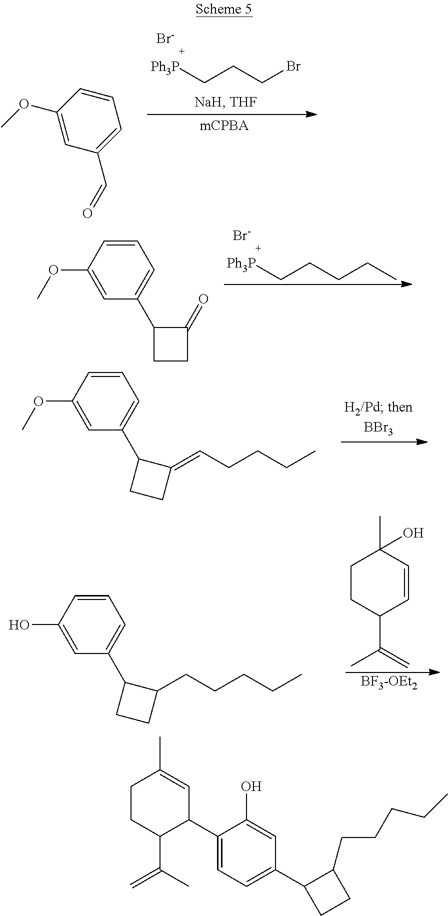

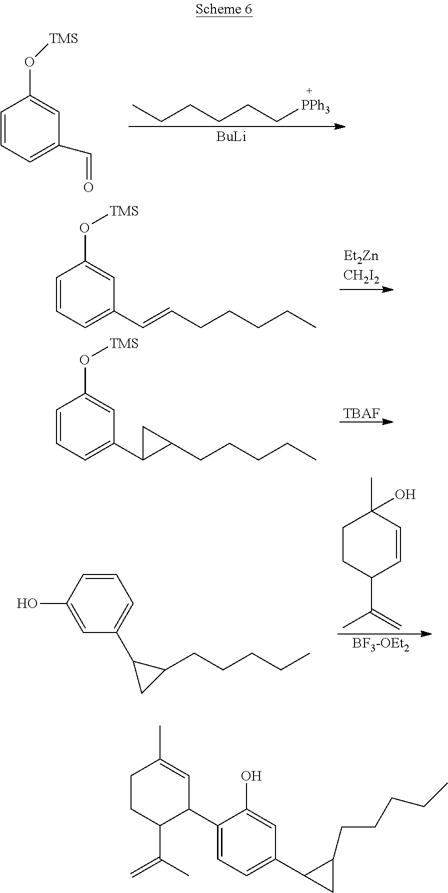

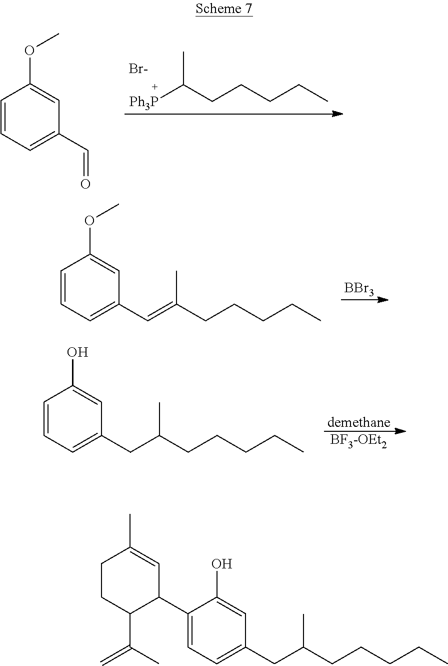

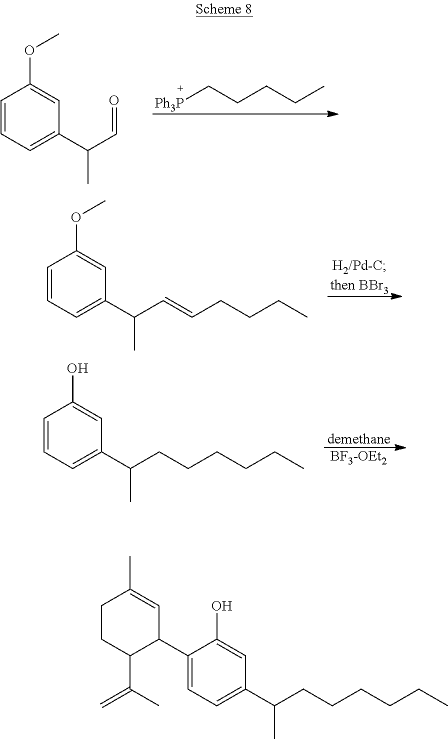

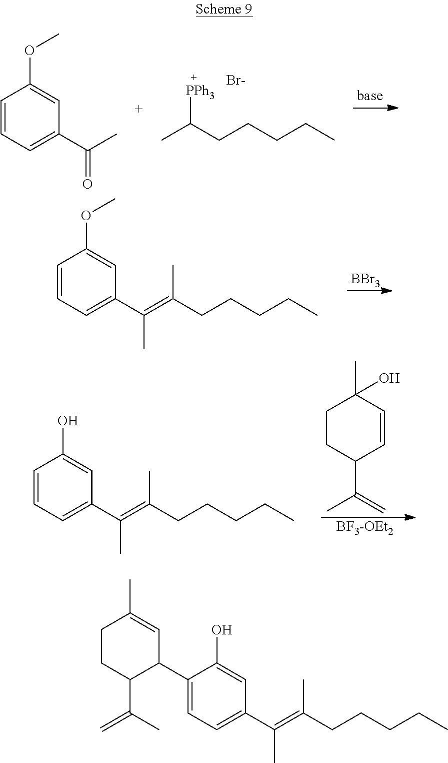

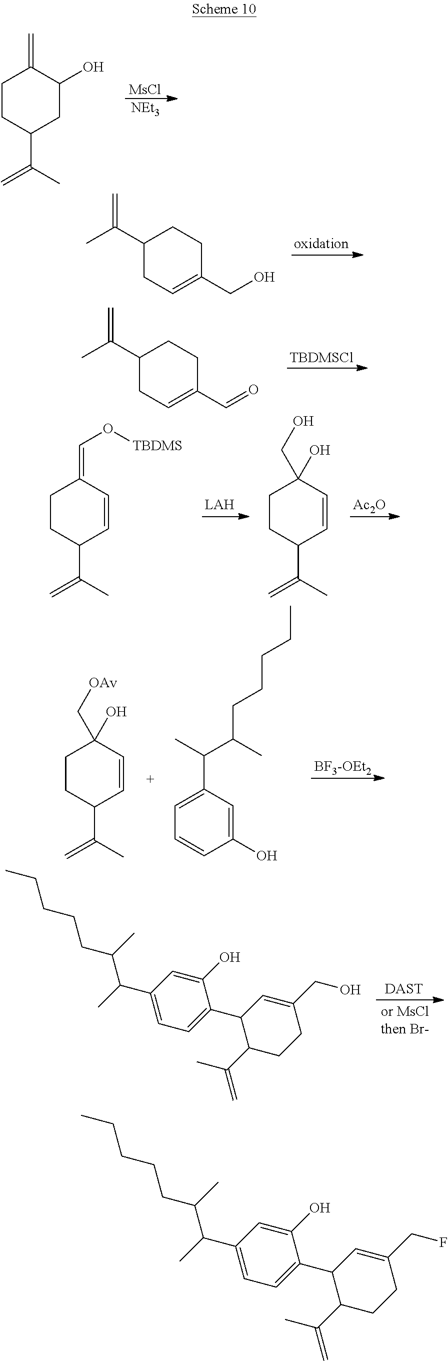

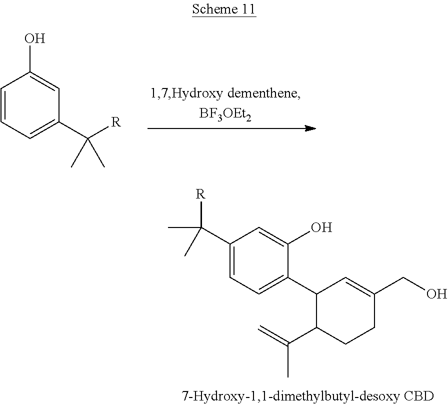

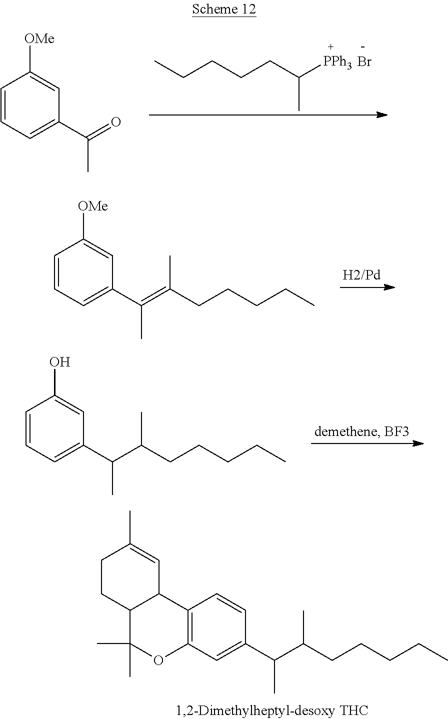

### REFERENCES

Kinney, W. A.; McDonnell, M. E.; Zhong, H. M.; Liu, C.; Yang, L.; Ling, W.; Qian, T.; Chen, Y.; Cai, Z.; Petkanas, D.; Brenneman, D. E., Discovery of KLS-13019, a cannabidiol-derived neuroprotective agent, with improved potency, safety, and permeability. ACS Med Chem Lett., 2016, 7(4), 424-428
Pop, E.; Rachwal, S.; Vlasak, J.; Biegon, A.; Zharikova, A.; Prokai, L., In vitro and in vivo study of water-soluble prodrugs of dexanabinol. J Pharm Sci., 1999, 88(11), 1156-1160
Xiong, W.; Cheng, K.; Cui, T.; Godlewski, G.; Rice, K.; Xu, Y.; Zhang, L., Cannabinoid potentiation of glycine receptors contributes to cannabis-induced analgesia. Nat Chem Biol. 2011, 7(5), 296-303
Xiong, W.; Cui, T.; Cheng, K.; Yang, F.; Chen, S. R.; Willenbring, D.; Guan, Y.; Pan, H. L.; Ren, K.; Xu, Y.; Zhang, L., Cannabinoids suppress inflammatory and neuropathic pain by targeting α3 glycine receptors. J. Exp. Med., 2012, 209(6), 1121-1134
Xiong, W.; Chen, S. R.; He, L.; Cheng, K.; Zhao, Y. L.; Chen, H/; Li, D. P.; Homanics, G. E.; Peever, J; Rice, K. C.; Wu, L. G.; Pan, H. L.; Zhang, L., Presynaptic glycine receptors as a potential therapeutic target for hyperekplexia disease. Nat Neurosci. 2014, 17(2), 232-239

1. A cannabinoid compound of the formula I: 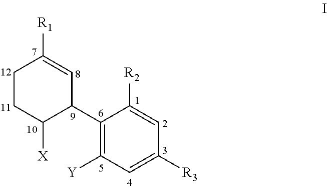
wherein:
X is the radical 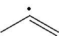
wherein . represents the point of attachment to formula I, and Y is H, —OH, —OR₄, or R₄; or
X is the radical 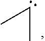
wherein . represents the point of attachment to formula I, and Y is —O—, and together with X and the carbon atoms to which they are attached form a dihydropyran ring,
or an enantiomer, diastereomer, racemate, or pharmaceutically acceptable salt thereof,
wherein:
R₁ is (C₁-C₃)alkyl, (C₁-C₃)haloalkyl, —(C₁-C₃)alkylene-OH, —(C₁-C₃)alkylene-COOH, —(C₁-C₃)alkylene-O—(C₁-C₁₂)alkyl, —(C₁-C₃)alkylene-O—C(O)—(C₁-C₁₂)alkyl, —(C₁-C₃)alkylene-C(O)—O—(C₁-C₁₂)alkyl, —COOH, R₆, or —(C₁-C₃)alkylene-R₆;
R₂ is H, —OH, —OR₄, or R₄;
R₃ is —OH, —OR₅, or R₅;
R₄ and R₅ each independently is (C₁-C₁₂)alkyl, (C₁-C₁₂)haloalkyl, (C₂-C₁₂)alkenyl, (C₂-C₁₂)alkynyl, (C₃-C₈)cycloalkyl, (C₃-C₈)cycloalkenyl, (C₃-C₈)cycloalkylene-(C₁-C₁₂)alkyl, (C₁-C₁₂)alkylene-(C₃-C₈)cycloalkyl, —C(O)—(C₁-C₁₂)alkyl, —C(O)—(C₁-C₁₂)haloalkyl, —C(O)—(C₂-C₁₂)alkenyl, —C(O)—(C₂-C₁₂)alkynyl, —C(O)—(C₃-C₈)cycloalkyl, —C(O)—(C₃-C₈)cycloalkenyl, non-aromatic (C₃-C₈)heterocyclyl, bridged (C₆-C₁₄)bicycloalkyl, bridged (C₈-C₁₆)tricycloalkyl, R₆, or the radical of the formula II: 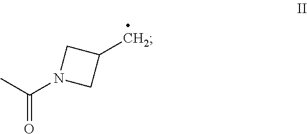
and
R₆ each independently is a drug selected from the group consisting of naproxen, ibuprofen, aspirin, betaine (trimethyl glycine), an opiate, an inducible nitric oxide synthase (iNOs) inhibitor, a poly(ADP-ribose) polymerase (PARP) inhibitor, efand a derivative thereof, linked directly or via a linker,
provided that:
(i) Y is H, but excluding the compound wherein R₂ is H; or wherein R₁ is CH₃, R₂ is —OH, and R₃ is n-pentyl; or
(ii) Y is —O—; and R₂ is H or R₄, but excluding the compound wherein R₁ is CH₃, R₂ is H, and R₃ is n-pentyl; or
(iii) Y is —OH, —OR₄, or R₄; R₂ is OH, —OR₄, or R₄; and
(a) R₁ is —(C₁-C₃)alkylene-R₆ or (b) R₂ is R₄ wherein R₄ is R₆ or (c) R₃ is R₅ wherein R₅ is R₆, or (d) Y is R₄ wherein R₄ is R₆.

2. The compound of claim 1, wherein:
(i) R₁ is (C₁-C₃)alkyl, (C₁-C₃)haloalkyl —(C₁-C₃)alkylene-OH, —(C₁-C₃)alkylene-O—C(O)—(C₁-C₁₂)alkyl, or —(C₁-C₃)alkylene-R₆; or
(ii) R₂ is H, —OH, —OR₄, or R₄; and R₄ is (C₁-C₁₂)alkyl, —C(O)—(C₁-C₁₂)alkyl, (C₃-C₈)cycloalkylene-(C₁-C₁₂)alkyl, R₆, or the radical of the formula II; or
(iii) R₃ is —OH, —OR₅, or R₅; and R₅ is (C₁-C₁₂)alkyl, —C(O)—(C₁-C₁₂)alkyl, (C₃-C₈)cycloalkylene-(C₁-C₁₂)alkyl, R₆, or the radical of the formula II.

3. The compound of claim 2, wherein:
(i) R₁ is —CH₃, —CH₂F, —CH₂—OH, —CH₂—O—C(O)—(C₁-C₁₂)alkyl, or —CH₂—R₆; or
(ii) R₂ is H, or —OH; or R₂ is —OR₄, and R₄ is —C(O)—(C₁-C₁₂)alkyl; or R₂ is R₄, and R₄ is R₆; or
(iii) R₃ is R₅; and R₅ is (C₁-C₁₂)alkyl, (C₃-C₈)cycloalkylene-(C₁-C₁₂)alkyl, R₆, or the radical of the formula II.

4-7. (canceled)

8. The compound of claim 1, wherein R₁ is (C₁-C₃)alkyl, (C₁-C₃)haloalkyl —(C₁-C₃)alkylene-OH, —(C₁-C₃)alkylene-O—C(O)—(C₁-C₁₂)alkyl, or —(C₁-C₃)alkylene-R₆; R₂ is H, —OH, —OR₄, or R₄; R₃ is —OH, —OR₅, or R₅; and R₄ and R₅ each independently is (C₁-C₁₂)alkyl, —C(O)—(C₁-C₁₂)alkyl, (C₃-C₈)cycloalkylene-(C₁-C₁₂)alkyl, R₆, or the radical of the formula II.

9. The compound of claim 8, wherein:
(i) R₁ is —CH₃, —CH₂F, —CH₂—OH, —CH₂—O—C(O)—(C₁-C₁₂)alkyl, or —CH₂—R₆; R₂ is H, or —OH; R₃ is R₅; R₅ is (C₁-C₁₂)alkyl, (C₃-C₈)cycloalkylene-(C₁-C₁₂)alkyl, R₆, or the radical of the formula II; and R₆ each independently is said drug linked directly or via a linker; or
(ii) R₁ is —CH₃, —CH₂F, —CH₂—OH, —CH₂—O—C(O)—(C₁-C₁₂)alkyl, or —CH₂—R₆; R₂ is —OR₄; R₃ is R₅; R₄ is —C(O)—(C₁-C₁₂)alkyl; R₅ is (C₁-C₁₂)alkyl, (C₃-C₈)cycloalkylene-(C₁-C₁₂)alkyl, R₆, or the radical of the formula II; and R₆ each independently is said drug linked directly or via a linker; or
(iii) R₁ is —CH₃, —CH₂F, —CH₂—OH, —CH₂—O—C(O)—(C₁-C₁₂)alkyl, or —CH₂—R₆; R₂ is R₄; R₃ is R₅; R₄ is R₆; R₅ is (C₁-C₁₂)alkyl, (C₃-C₈)cycloalkylene-(C₁-C₁₂)alkyl, R₆, or the radical of the formula II; and R₆ each independently is said drug linked directly or via a linker.

10-11. (canceled)

12. The compound of claim 1, wherein said opiate is codeine,
dihydrocodeine, diamorphine, buprenorphine, methadone, fentanyl, hydromorphone, oxycodone, pethidine, morphine, dextropropoxyphene, or tramadol; said PARP inhibitor is olaparib, veliparib, veliparib acetate, rucaparib, talazoparib, niraparib,
or said iNOs inhibitor is N-[[3-(Aminomethyl)phenyl]methyl]-ethanimidamide dihydrochloride (1400W), N⁶-(1-Iminoethyl)-L-lysine hydrochloride (L-NIL), N⁵-(1-Iminoethyl)-L-ornithine dihydrochloride (L-NIO), or (2S)-2-amino-4-[(2-ethanimidamidoethyl)sulfanyl]butanoic acid (GW274150).

13. The compound of claim 1, wherein said linker each independently is of the formula —O—C(O)—(CH₂)ₙ—C(O)—O—CH₂—, or —O—C(O)—(CH₂)ₙ—C(O)—O—, wherein n is an integer of 1-8.

14. The compound of claim 1, wherein (a) R₁ is —(C₁-C₃)alkylene-R₆; or (b) R₂ is R₄; and R₄ is R₆; or (c) R₃ is R₅; and R₅ is R₆; or (d) Y is R₄; and R₄ is R₆.

15. The compound of claim 1, wherein Y is H.

16. The compound of claim 15, wherein (i) R₂ is —OH; (ii) R₂ is —OR₄; and R₄ is —C(O)—(C₁-C₁₂)alkyl; or (iii) R₂ is R₄; and R₄ is (C₁-C₁₂)alkyl, R₆, or the radical of the formula II.

17. The compound of claim 16, wherein R₁ is —CH₃, —CH₂F, —CH₂—OH, —CH₂—O—C(O)—(C₁-C₁₂)alkyl, or —CH₂—R₆; R₃ is R₅; and R₅ is (C₁-C₁₂)alkyl, (C₃-C₈)cycloalkylene-(C₁-C₁₂)alkyl, R₆, or the radical of the formula II.

18. The compound of claim 1, wherein Y is —OH, —OR₄, or R₄ wherein R₄ is R₆.

19. The compound of claim 18, wherein (i) R₂ is —OH; (ii) R₂ is —OR₄; and R₄ is —C(O)—(C₁-C₁₂)alkyl; or (iii) R₂ is R₄; and R₄ is (C₁-C₁₂)alkyl, R₆, or the radical of the formula II.

20. The compound of claim 19, wherein R₁ is —CH₃, —CH₂F, —CH₂—OH, —CH₂—O—C(O)—(C₁-C₁₂)alkyl, or —CH₂—R₆; R₃ is R₅; and R₅ is (C₁-C₁₂)alkyl, (C₃-C₈)cycloalkylene-(C₁-C₁₂)alkyl, R₆, or the radical of the formula II.

21. The compound of claim 1, wherein Y is —O— and together with X and the carbon atoms to which they are attached form a dihydropyran ring.

22. The compound of claim 21, wherein (i) R₂ is H; or (ii) R₂ is R₄; and R₄ is (C₁-C₁₂)alkyl, R₆, or the radical of the formula II.

23. The compound of claim 22, wherein R₁ is —CH₃, —CH₂F, —CH₂—OH, —CH₂—O—C(O)—(C₁-C₁₂)alkyl, or —CH₂—R₆; R₃ is R₅; and R₅ is (C₁-C₁₂)alkyl, (C₃-C₈)cycloalkylene-(C₁-C₁₂)alkyl, R₆, or the radical of the formula II.

24. The compound of claim 15, wherein:
(i) R₁ is —CH₃; R₂ is —OH; R₃ is R₅; and R₅ is 2-methyloctan-2-yl, 3-methyloctan-2-yl, 2-methylpentan-2-yl, 3-methylhexan-2-yl, 3-methylheptan-2-yl, 3-methylnonan-2-yl, octan-2-yl; 2-methylheptyl; 3-methyloct-2-en-2-yl, 2-pentylcyclopropyl, 2-pentylcyclobutyl, 1-methyl-2-pentylcyclopropyl, or the radical of the formula II;
(ii) R₁ is —CH₂F; R₂ is —OH; R₃ is R₅; and R₅ is 3-methyloctan 2 yl;
(iii) R₁ is —CH₃; R₂ is R₄; R₃ is R₅; R₄ is R₆; R₅ is pentyl, 2-methyloctan-2-yl, 3-methyloctan-2-yl, or the radical of the formula II; and R₆ is naproxen linked through the carboxyl group thereof;
(iv) R₁ is —CH₂—OH; R₂ is —OH; R₃ is R₅; and R₅ is pentyl, 2-methyloctan-2-yl, 3-methylpentane-2-yl, 3-methyloctan-2-yl, or 2-methylbutan 2 yl;
(v) R₁ is —CH₂—OH; R₂ is R₄; R₃ is R₅; R₄ is R₆; R₅ is pentyl, or 2-methyloctan-2-yl; and R₆ is naproxen linked through the carboxyl group thereof;
(vi) R₁ is —CH₂—R₆; R₂ is —OH; R₃ is R₅; and R₅ is pentyl, 2-methyloctan-2-yl, 3-methyloctan-2-yl, or the radical of the formula II; and R₆ is betaine linked through the carboxyl group thereof;
(vii) R₁ is —CH₂—R₆; R₂ is —OH; R₃ is R₅; R₅ is pentyl; and R₆ is naproxen linked through the carboxyl group thereof;
(viii) R₁ is —CH₂—R₆ wherein R₆ is betaine linked through the carboxyl group thereof; R₂ is R₄; R₃ is R₅; R₄ is R₆ wherein R₆ is naproxen linked through the carboxyl group thereof; and R₅ is pentyl, 2-methyloctan-2-yl, or 3-methyloctan 2 yl; or
(ix) R₁ is —CH₂—R₆ wherein R₆ is naproxen linked through the carboxyl group thereof; R₂ is R₄; R₃ is R₅; R₄ is R₆ wherein R₆ is betaine linked through the carboxyl group thereof; and R₅ is pentyl, or 2-methyloctan 2 yl.

25. The compound of claim 18, wherein:
(i) Y is —OH; R₁ is CH₃; R₂ is —OH; R₃ is R₅; R₅ is R₆; and R₆ is veliparib or a derivative thereof, linked directly through the methyl group thereof;
(ii) Y is —OH; R₁ is —CH₃; R₂ is —OH; R₃ is R₅; R₅ is R₆; and R₆ is PJ34 linked through the dimethylamino group thereof and via a linker of the formula —CH₂—O—C(O)—(CH₂)ₙ—C(O)—O—, wherein n is an integer of 1-3;
(iii) Y is —OH; R₁ is —CH₃; R₂ is —OH; R₃ is R₅; R₅ is R₆; and R₆ is niraparib linked through the nitrogen atom of the piperidine ring and via a linker of the formula —CH₂—O—C(O)—(CH₂)ₙ—C(O)—O—, wherein n is an integer of 1-3;
(iv) Y is —OH; R₁ is —CH₂—R₆; R₂ is —OH; R₃ is R₅; R₅ is pentyl; and R₆ is codeine linked through the nitrogen atom thereof and via a linker of the formula —CH₂—O—C(O)—(CH₂)ₙ—C(O)—O—, wherein n is an integer of 1-3;
(v) Y is —OH; R₁ is —CH₂—R₆; R₂ is —OH; R₃ is R₅; R₅ is pentyl; and R₆ is PJ34 linked through the dimethylamino group thereof and via a linker of the formula —CH₂—O—C(O)—(CH₂)ₙ—C(O)—O—, wherein n is an integer of 1-3;
(vi) Y is —OH; R₁ is —CH₂—R₆; R₂ is —OH; R₃ is R₅; R₅ is pentyl; and R₆ is niraparib linked through the nitrogen atom of the piperidine ring and via a linker of the formula —CH₂—O—C(O)—(CH₂)ₙ—C(O)—O—, wherein n is an integer of 1-3;
(vii) Y is —OH; R₁ is CH₃; R₂ is R₄; R₃ is R₅; R₄ is R₆; R₅ is pentyl; and R₆ is codeine linked through the nitrogen atom thereof and via a linker of the formula —CH₂—O—C(O)—(CH₂)ₙ—C(O)—O—, wherein n is an integer of 1-3;
(viii) Y is —OH; R₁ is —CH₃; R₂ is R₄; R₃ is R₅; R₄ is R₆; R₅ is pentyl; and R₆ is PJ34 linked through the dimethylamino group thereof and via a linker of the formula —CH₂—O—C(O)—(CH₂)ₙ—C(O)—O—, wherein n is an integer of 1-3;
(ix) Y is —OH; R₁ is —CH₃; R₂ is R₄; R₃ is R₅; R₄ is R₆; R₅ is pentyl; and R₆ is niraparib linked through the nitrogen atom of the piperidine ring and via a linker of the formula —CH₂—O—C(O)—(CH₂)ₙ—C(O)—O—, wherein n is an integer of 1-3;
(x) Y is R₄ wherein R₄ is R₆ and R₆ is betaine linked through the carboxyl group thereof; R₁ is CH₃; R₂ is R₄; R₃ is R₅; R₄ is R₆ wherein R₆ is betaine linked through the carboxyl group thereof; and R₅ is R₆ wherein R₆ is veliparib or a derivative thereof, linked directly through the methyl group thereof (herein; or
(xi) Y is R₄; R₁ is —CH₃; R₂ is R₄; R₃ is R₅; R₄ is R₆; R₅ is pentyl; and R₆ each is codeine linked through the nitrogen atom thereof and via a linker of the formula —CH₂—O—C(O)—(CH₂)ₙ—C(O)—O—, wherein n is an integer of 1-3.

26. The compound of claim 21, wherein:
(i) R₁ is —CH₃; R₂ is H; R₃ is R₅; and R₅ is 3-methylpctan-2-yl, 2-methyloctan-2-yl, or 2-methylpentan 2 yl;
(ii) R₁ is —CH₂—OH; R₂ is H; R₃ is R₅; and R₅ is pentyl, or 2-methylpentan-2-yl;
(iii) R₁ is —CH₃; R₂ is R₄; R₃ is R₅; R₄ is R₆; R₅ is propyl; and R₆ is naproxen linked through the carboxyl group thereof; or
(iv) R₁ is —CH₂—R₆ wherein R₆ is betaine linked through the carboxyl group thereof; R₂ is R₄; R₃ is R₅; R₄ is R₆ wherein R₆ is naproxen linked through the carboxyl group thereof; and R₅ is propyl.

27-29. (canceled)

30. A pharmaceutical composition comprising a compound of claim 1, or an enantiomer, diastereomer, racemate, or pharmaceutically acceptable salt thereof, and a pharmaceutically acceptable carrier.

31. The pharmaceutical composition of claim 30, for intravenous, intraarterial, intramuscular, intraperitoneal, intrathecal, intrapleural, intratracheal, subcutaneous, topical, inhalational, or oral administration.

32-34. (canceled)

35. A method for providing neuroprotection, treating pain, or treating a disease associated with glycine receptor (GlyR) deficiency selected from hyperekplexia disease, in an individual in need thereof, comprising administering to said individual an effective amount of a compound according to claim 1, or an enantiomer, diastereomer, racemate, or pharmaceutically acceptable salt thereof.
